(19) United States
(12) Patent Application Publication
(10) Pub. No.: US 2026/0200907 A1
(43) Pub. Date: Jul. 16, 2026

(54) CRYSTALLINE FORMS OF SPLEEN TYROSINE KINASE INHIBITOR SKI-O-703

(71) Applicant: Oscotec Inc., Seongnam-si (KR)

(72) Inventors: Pingyun Chen, Carmel, IN (US); Min-Woo Kim, Montville, NJ (US); Song-Eun Park, Gyeonggi-do (KR); Dong-Sik Jung, Chungcheongnam-do (KR); Yutae Kim, Gyeonggi-do (KR); Young Chun Jung, Bedford, MA (US)

(21) Appl. No.: 19/440,242

(22) Filed: Jan. 5, 2026

Related U.S. Application Data
(60) Provisional application No. 63/742,097, filed on Jan. 6, 2025.

(51) Int. Cl. C07D 413/14; A61K 31/506; C07C 309/04

(52) U.S. Cl. CPC C07D 413/14; A61K 31/506; C07C 309/04; C07B 2200/13

(57) ABSTRACT

Disclosed are crystalline forms of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (SKI-O-703, “Compound 1”), their process of manufacture, pharmaceutical compositions, and methods for using the inventive crystal forms for the treatment of diseases such as cancer, autoimmune diseases, neurodegenerative diseases, and disorders associated with the dysfunction of tyrosine kinase.

This application claims the benefit of priority to U.S. Provisional Patent Application No. 63/742,097 filed on Jan. 6, 2025.

### BACKGROUND

Spleen tyrosine kinase (SYK) is a cytoplasmic non-receptor tyrosine kinase and plays a central role in the regulation of various signal transduction pathways. Even though the gene of SYK was first designated using a spleen cDNA library, this kinase is mainly expressed in the cells of hematopoietic origin, and effectively transmits different signals from T-cell antigen receptors (TCR) and B-cell antigen receptors (BCR).

In recent years, SYK expression was found in non-hematopoietic tissues, e.g., vascular smooth muscle, normal mammary gland, and pulmonary tissue. Therefore, diverse biological functions, such as immune recognition and inflammatory response, can be effectively regulated by SYK, and SYK is also considered as a novel therapy target for non-hematopoietic diseases (e.g., liver fibrosis, lung cancer, and diabetic cardiomyopathy).

SYK is also implicated in the pathogenesis of the autoimmune disease immune thrombocytopenia (ITP). SYK inhibitors are being evaluated as potential therapeutics for the treatment of neurodegenerative diseases, cancer, COPD, rheumatoid arthritis, osteopenia, osteoporosis, asthma, and allergic rhinitis.

One potent inhibitor of SYK is cevidoplenib bismesylate (SKI-O-703, “Compound 1”). Chemical methods for synthesizing small molecule inhibitors of SYK, including Compound 1, are disclosed in WO 2011/060292 and WO 2015/061369 which are incorporated herein in their entirety.

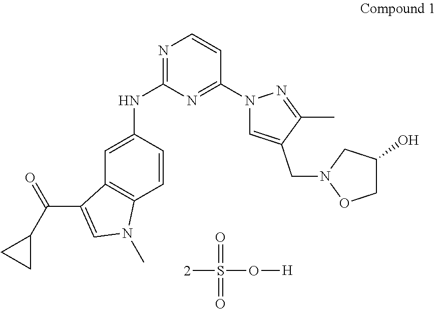

### SUMMARY

The present invention relates to the discovery of crystalline forms of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (SKI-O-703, Compound 1), which are obtained in high yield and high purity compared to amorphous Compound 1.

In one embodiment, the present disclosure provides a monohydrate crystalline Form J of Compound 1. Form J is characterized by an X-ray powder diffraction (XRPD) pattern comprising signals at 14.1, 16.9, 18.1, 19.9, 15.3, and 26.5 2θ±0.2 °2θ as determined on a diffractometer using Cu-Kα radiation at a wavelength of 1.54 Å.

In one embodiment, Form J of Compound 1 is characterized by an XRPD pattern that is substantially as shown in FIG. 13.

In one embodiment, Form J of Compound 1 is characterized by a thermogravimetric analysis (TGA) thermogram comprising a weight loss of about 2.6% between 3° and 100° C. In one embodiment, the TGA thermogram for Form J is substantially as shown in FIG. 14.

In one embodiment, Form J of Compound 1 comprises between 0.01 ppm to 0.9 ppm of

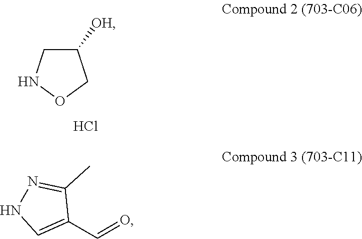

and combinations thereof.

In another embodiment, Form J of Compound 1 comprises Compound 4 (703-C09) in a weight percentage amount of about 0.05% to about 0.25%.

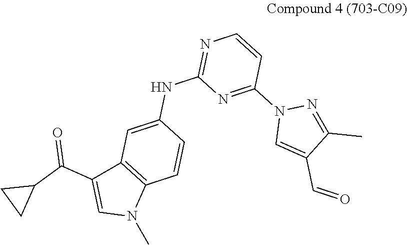 [SMILES: Cc1nn(-c2ccnc(Nc3ccc4c(c3)c(C(=O)C3CC3)cn4C)n2)cc1C=O]

In another embodiment, there is provided a monohydrate crystalline Form F of Compound 1, comprising XRPD signals at 5.6, 16.7, 18.9, 20.3, 24.0, and 29.3 °2θ±0.2 °2θ as determined using a diffractometer using Cu-Kα radiation at a wavelength of 1.54 Å. In one embodiment, the XRPD pattern for Form F is substantially as shown in FIG. 9.

In one embodiment, Form F is further characterized by a thermogravimetric analysis (TGA) thermogram comprising a weight loss of about 2.6% between 3° and 100° C. In another embodiment, the TGA thermogram of Form F is substantially as shown in FIG. 10.

In one embodiment, the disclosure provides a partial hydrate crystalline Form N of Compound 1, characterized by an X-ray powder diffraction (XRPD) pattern comprising signals at 14.5, 16.7, 17.1, 19.4, 20.2, 24.1, and 25.7° 2θ±0.2 ° 2θ as determined using a diffractometer using Cu-Kα, radiation at a wavelength of 1.54 Å.

In an additional embodiment, the XRPD pattern of Form N is substantially as shown in FIG. 19.

In one embodiment, Form N of Compound 1 is characterized by a thermogravimetric analysis (TGA) thermogram comprising a weight loss of about 1.0% between 3° and 100° C. In one embodiment, the TGA thermogram of Form N is substantially as shown in FIG. 20.

In one embodiment, the present disclosure provides an anhydrate crystalline Form M of Compound 1, characterized by an X-ray powder diffraction (XRPD) pattern comprising signals at 26.0, 23.5, 20.4, 19.9, 18.2, 16.3 °2θ±0.2 °2θ as determined using a diffractometer using Cu-Kα radiation at a wavelength of 1.54 Å. In another embodiment, the XRPD pattern is substantially as shown in FIG. 18.

In an embodiment, there is provided crystalline Form E of Compound 1 that is characterized by an X-ray powder diffraction (XRPD) pattern substantially as shown in FIG. 7. In one embodiment, Form E is further characterized by a TGA thermogram comprising a weight loss of about 1.8% between 3° and 100° C. In one embodiment, Form E is further characterized by a TGA thermogram substantially as shown in FIG. 8.

According to another embodiment, the present disclosure provides a pharmaceutical composition comprising (i) a therapeutically effective amount of any one of Form J, Form F, Form N, Form M, and Form E of Compound 1 as described herein and (ii) at least one solid pharmaceutically acceptable excipient.

In various embodiments, the pharmaceutical composition comprises two or more different crystalline forms as described herein to form binary or ternary compositions. In exemplary embodiment, the pharmaceutical composition comprises a mixture of Form J and Form N, or Form J and Form F, or Form F and Form N, or Form J and Form M, Form F and Form M, or Form N and Form M. The ternary composition comprises any combination and amount of any three crystal Forms as disclosed herein.

In another embodiment, the disclosure is drawn to a method for treating a subject suffering from idiopathic thrombocytopenic purpura (ITP), rheumatoid arthritis (RA), warm autoimmune hemolytic anemia (wAIHA), systemic lupus erythematosus, Psoriasis, or antiphospholipid syndrome (APS) The method comprises administering to the subject a therapeutically effective amount of any one of Forms J, F, N, M, and E of Compound 1 as described herein.

According to another embodiment, the disclosure is drawn to use of any one of Forms J, F, N, M, and E of Compound 1 for treating ITP in a subject suffering therefrom.

In one embodiment, the disclosure is drawn to a method for synthesizing crystalline Form J of Compound 1, by
(a) dissolving cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone (SKI-O-592) in warm DMSO whereby a solution is formed;
(b) contacting the solution from (a) with a DMSO solution of methanesulfonic acid whereby a second solution is formed;
(c) adding water and then acetone to the second solution from (b) whereby a third solution is formed; and
(d) cooling the third solution from (c) whereby Form J is formed.

### BRIEF DESCRIPTION OF FIGURES

FIG. 1: X-ray powder diffraction (XRPD) pattern of Form A.

FIG. 2: TGA thermogram of Form A.

FIG. 3: X-ray powder diffraction (XRPD) pattern of Form B.

FIG. 4: TGA thermogram of Form B.

FIG. 5: X-ray powder diffraction (XRPD) pattern of Form C.

FIG. 6: TGA thermogram of Form C.

FIG. 7: X-ray powder diffraction (XRPD) pattern of Form E.

FIG. 8: TGA thermogram of Form E.

FIG. 9: X-ray powder diffraction (XRPD) pattern of Form F.

FIG. 10: TGA thermogram of Form F.

FIG. 11: X-ray powder diffraction (XRPD) pattern of Form H.

FIG. 12: TGA thermogram of Form H.

FIG. 13: X-ray powder diffraction (XRPD) pattern of Form J.

FIG. 14: TGA thermogram of Form J.

FIG. 15: X-ray powder diffraction (XRPD) pattern of Form L.

FIG. 16: TGA thermogram of Form L.

FIG. 17: Thermal ellipsoid plot of single crystal structure of Form M.

FIG. 18: X-ray powder diffraction (XRPD) pattern of Form M.

FIG. 19: X-ray powder diffraction (XRPD) pattern of Form N.

FIG. 20: TGA thermogram of Form N.

FIG. 21: X-ray powder diffraction (XRPD) pattern of amorphous SKI-O-703.

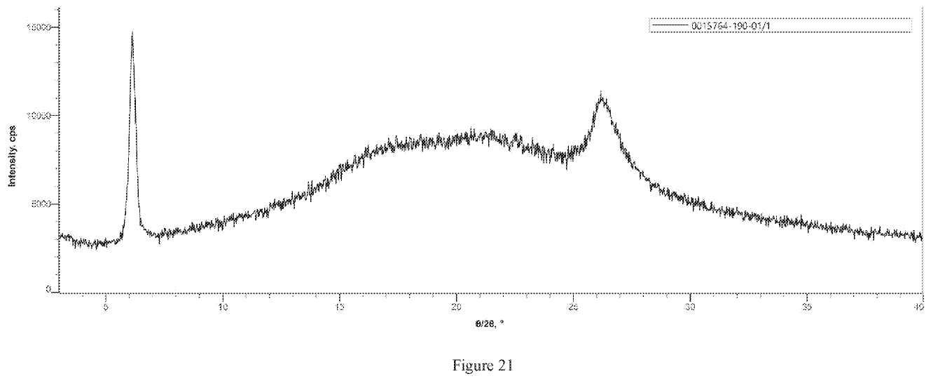

FIG. 22: X-ray powder diffraction (XRPD) pattern of SKI-O-592.

FIG. 23: X-ray powder diffraction (XRPD) pattern of Form J following impurity spiking studies.

FIG. 24: X-ray powder diffraction (XRPD) pattern of Form F following impurity spiking studies.

FIG. 25: Mean (±SD, n=3) Plasma Concentration Vs Time Curves of freebase of SKI-O-703 after administrated of SKI-O-703 via Oral Capsule in Male SD Rats.

FIG. 26: SKI-O-703 monohydrate Form J crystal packing shows a unit cell and a channel-like structure along the [1,0,0]direction.

FIG. 27: Variable Humidity XRPD of Form J and its related forms (Form N input).

FIG. 28: Dynamic Vapor Sorption Isotherm Profiles for Forms J, M and N.

### DETAILED DESCRIPTION

### Definitions

Abbreviations and terms used throughout the specification have the following meanings.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the term.

The term “inhibitor” refers to a compound which inhibits one or more kinases. For example, the phrase “SYK inhibitor” refers to a compound which inhibits the SYK receptor and reduces signaling.

The phrases “pharmaceutically acceptable formulation” or “pharmaceutically acceptable composition” refer to a mixture of a crystalline form described herein along with other chemical agents, such as carriers, excipients, suspending agents, lubricants, stabilizers, dispersing agents, pH balancing agents, diluents, and/or thickening agents.

The phrase “therapeutically effective amount” refers to any amount of a compound which when administered to a subject and compared to a subject receiving a placebo, results in improved treatment, healing, prevention, or amelioration of a disease or disorder, side effect, and/or reduces the rate of advancement of a disease or disorder.

As used herein, the terms “treat” and “treatment” refer to methods of alleviating, abating, or ameliorating a disease, symptoms of a disease/condition, or preventing additional symptoms from developing.

The terms “subject” and “patient” are used interchangeably, and refer to a mammal or non-mammal. For example, mammals include human, dogs, cats, mice, monkeys, rats, rabbits, horses, cows, guinea pigs, and sheep.

The phrase “pharmaceutical carrier” refers to chemical compounds or agents that facilitate the incorporation of a crystalline form described herein into cells or tissues.

The term “pharmaceutical diluent” refers to chemical compounds or agents that are used to dilute a crystalline form described herein prior to delivery. Diluents can also be used to stabilize crystalline forms described herein.

### Crystalline Forms

### Form A

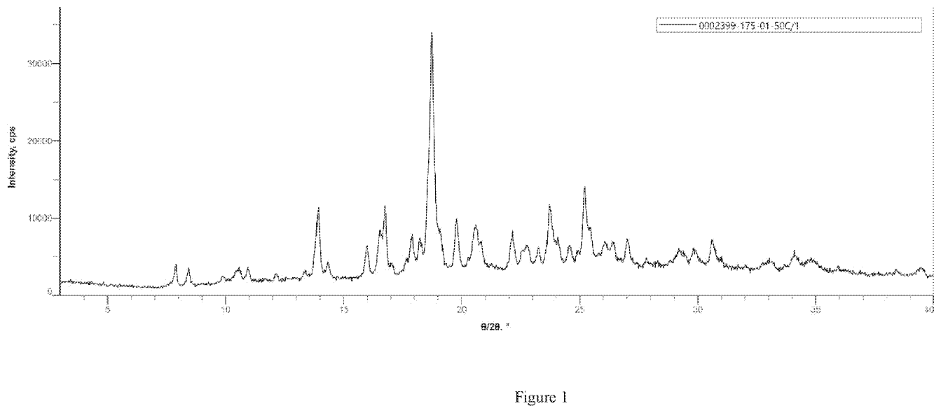

In one embodiment there is provided Form A, a crystalline form of Compound 1. Form A is characterized by an X-ray powder diffractogram pattern comprising signals listed in Table 11 and shown in FIG. 1.

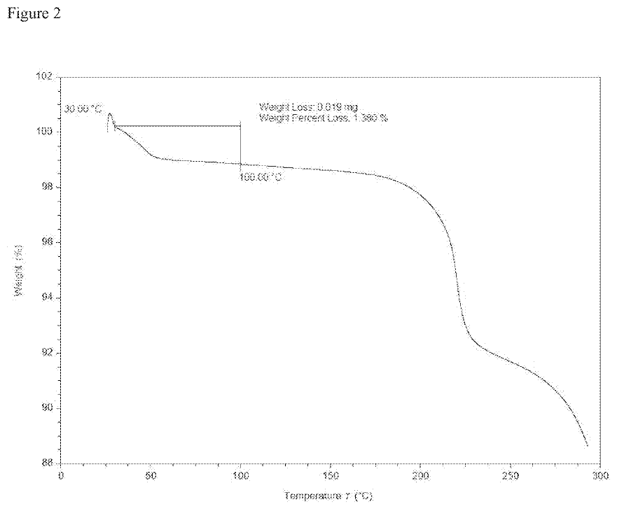

The TGA thermogram for Form A (FIG. 2) comprises a weight loss step that begins at about 30° C. and concludes before 100° C., constituting a mass loss of about 1.4% and a steep decrease above 180° C. most likely attributed to decomposition of the crystalline form. The disclosure further embodies a process for the synthesis of Form A crystal, as described below in Example 1. ¹H NMR of the crystalline form showed no residual solvent and 2 equivalents of the mesylate anion. Also provided in an embodiment is a synthesis of Form A crystals as described in Example 1.

### Form B

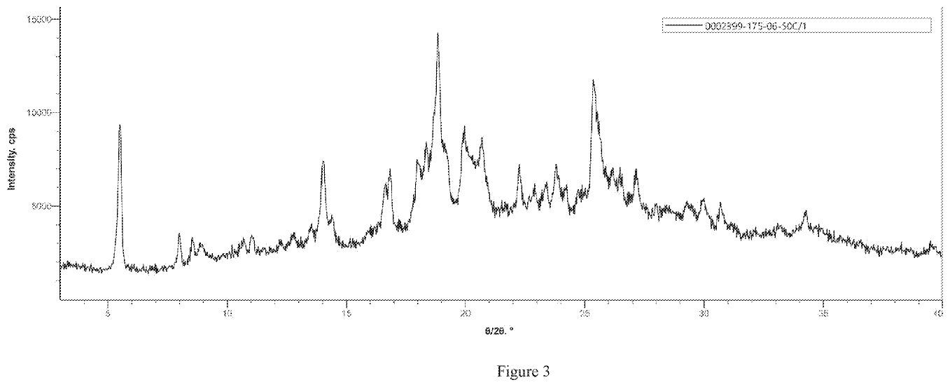

In one embodiment, the disclosure provides crystalline Form B of Compound 1, characterized by an X-ray powder diffractogram comprising signals listed in Table 13 below, and as substantially shown in FIG. 3.

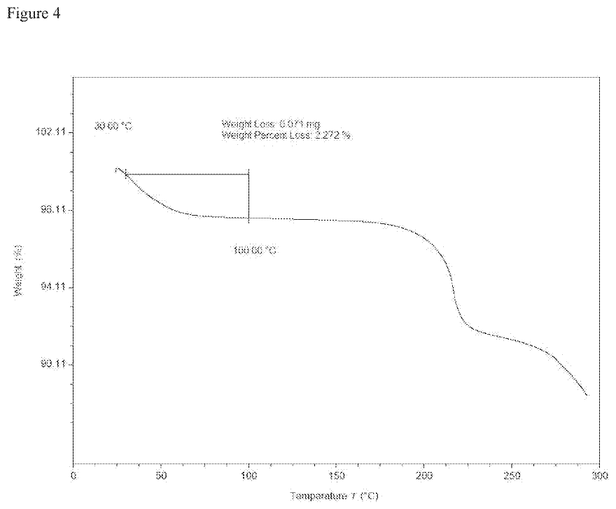

The TGA thermogram for Form B (FIG. 4) comprises a weight loss step that begins at about 30° C. and concludes before 100° C. and a mass loss of about 2.5% and a second weight loss step (steep decrease). Also provided in an embodiment is a process for the synthesis of Form B, as described below in Example 3, with ¹H NMR of the crystalline form showing no residual solvent.

### Form C

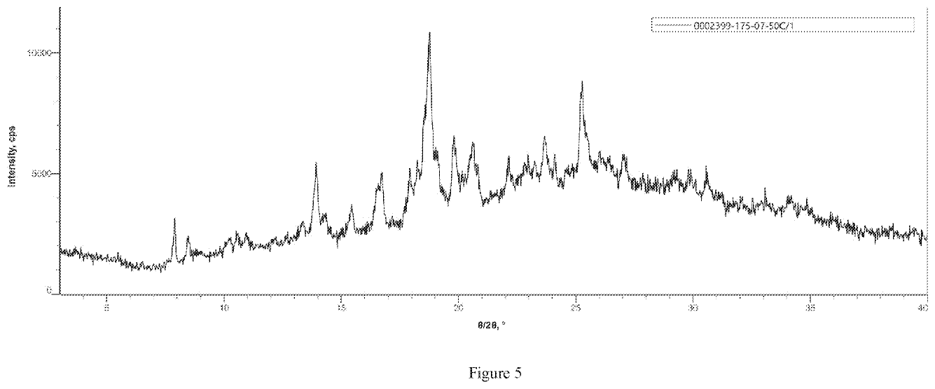

In one embodiment, the disclosure provides crystalline Form C of Compound 1, characterized by an X-ray powder diffractogram comprising signals listed in Table 14. In a further embodiment, Form C is characterized by its X-ray powder diffractogram substantially as shown in FIG. 5.

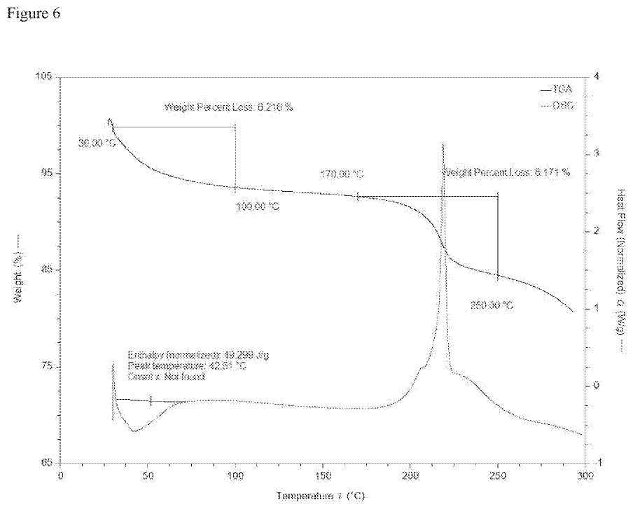

The TGA thermogram for Form C (FIG. 6) comprises a weight loss step that begins at about 30° C. and concludes before 100° C. with a mass loss of about 6.2%. Also provided in an embodiment is a process for the synthesis of crystalline Form C, as described below in Example 4, with ¹H NMR of the crystalline form showing about 0.7% 1,4-dioxane present as residual solvent.

### Form E

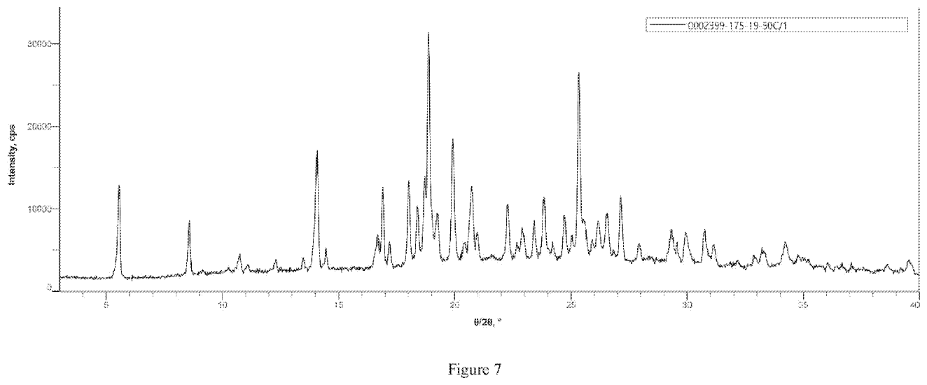

In one embodiment, the disclosure provides crystalline Form E of Compound 1, characterized by an X-ray powder diffractogram comprising signals listed in Table 15. In a further embodiment, Form E is characterized by its X-ray powder diffractogram substantially as shown in FIG. 7.

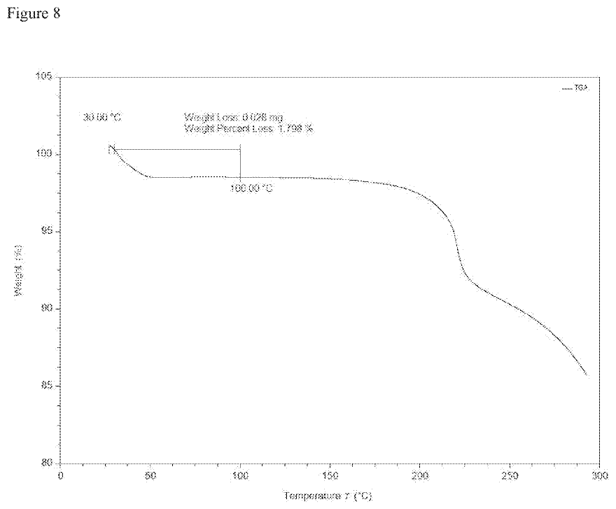

The TGA thermogram for Form E (FIG. 8) comprises a weight loss step that begins at about 30° C. and concludes before 100° C. with a mass loss of about 1.8%. Also provided in an embodiment isa process for the synthesis of crystalline Form E, as described below in Example 5, with ¹H NMR of the crystalline Form E showing no residual solvent.

### Form F

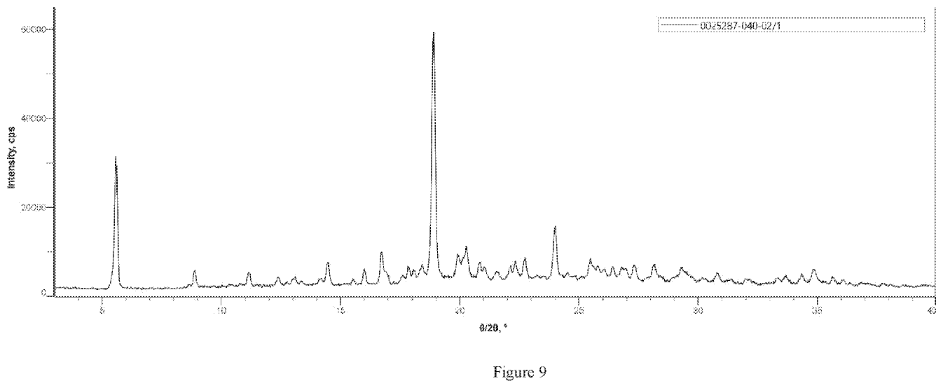

In one embodiment, the disclosure provides crystalline Form F of Compound 1, characterized by an X-ray powder diffractogram comprising signals as listed in Table 16, such as at 5.6, 16.7, 18.9, 20.3, 24.0, and 29.3 °2θ±0.2 °2θ. Additionally, Form F is further characterized by signals at 13.4 and 21.5 °2θ±0.2 °2θ. In a further embodiment, Form F is characterized by its X-ray powder diffractogram substantially as shown in FIG. 9.

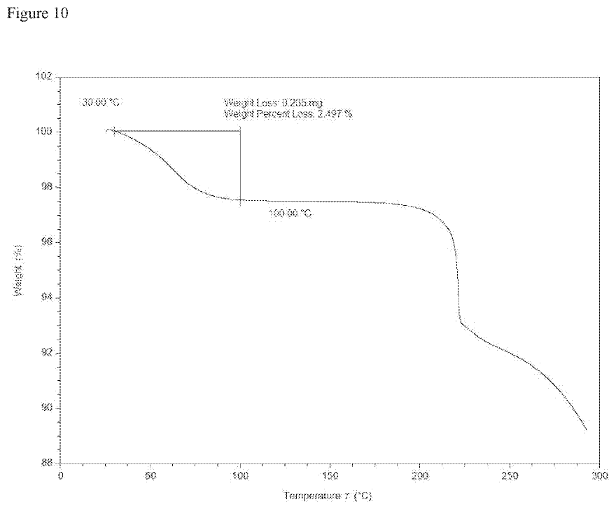

The TGA thermogram for Form F (FIG. 10) comprises a weight loss step that has no discernible onset point and concludes before 100° C. with a mass loss of about 2.6%, followed by a steep decrease at a temperature above 200° C. Also provided in an embodiment isa process for the synthesis of crystalline Form F, as described below in Example 6, with ¹H NMR of the crystalline Form F showing no residual solvent.

### Form H

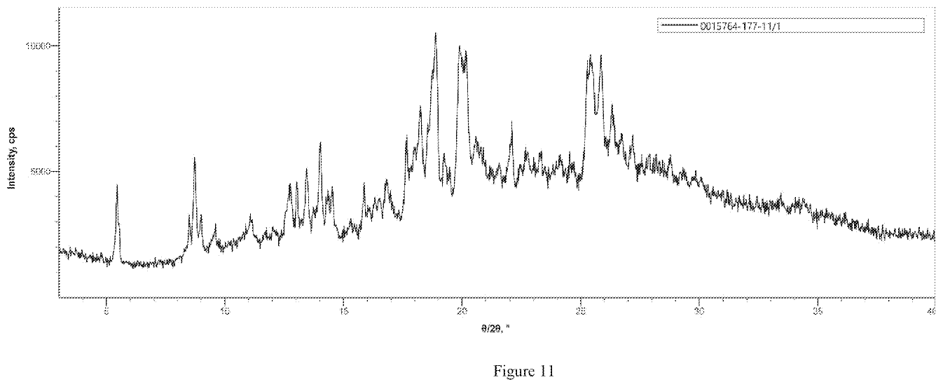

In one embodiment, the disclosure provides crystalline Form H of Compound 1, characterized by an X-ray powder diffractogram comprising signals listed in Table 18. In a further embodiment, Form H is further characterized by its X-ray powder diffractogram substantially as shown in FIG. 11. In one embodiment, Form H is obtained from Form F using a MeOH/H₂O (95/5) solvent system and a slurry ripening process. Form H was also obtained from Form F using MeOH/H₂O (9/1) or THF/H₂O (9/1) solvent systems, and via slow evaporation.

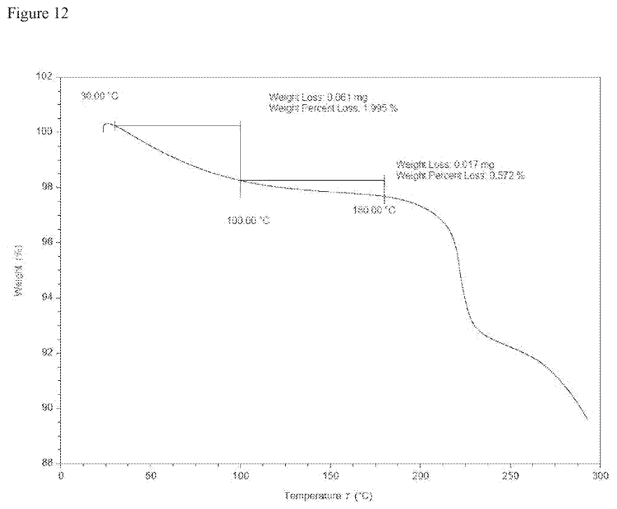

The TGA thermogram for Form H (FIG. 12) comprises a weight loss step that has no discernible onset point and concludes before 100° C. with a mass loss of about 2.1% as observed in FIG. 12. ¹H NMR of the crystalline Form H showed no solvent residue.

### Form J

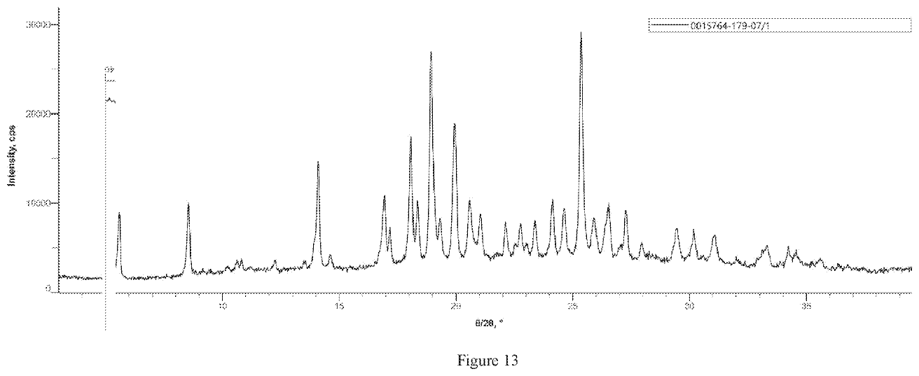

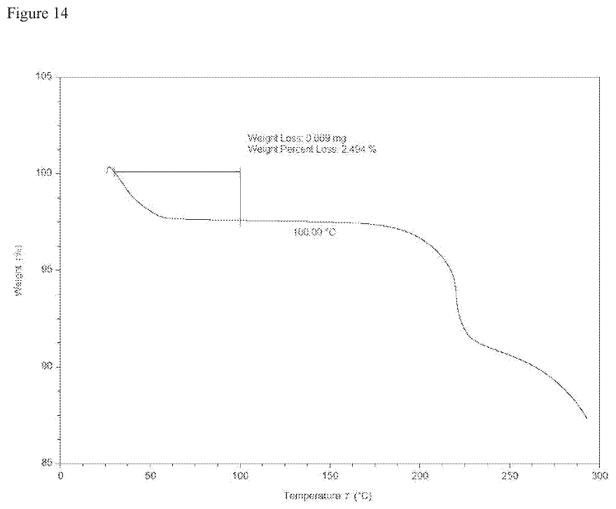

In other embodiments is crystalline Form J of Compound 1, characterized by an X-ray powder diffractogram comprising signals at 14.1, 16.9, 18.1, 19.9, 25.3, and 26.5 2θ±0.2 °2θ as determined on a diffractometer using Cu-Kα radiation at a wavelength of 1.54 Å. In a further embodiment, Form J is characterized by its X-ray powder diffractogram substantially as shown in FIG. 13. In one embodiment, the disclosure provides crystalline Form J characterized by an X-ray powder diffractogram comprising signals as listed in Table 19. The TGA thermogram for Form J (FIG. 14) comprises a weight loss step that begins at about 30° C. and concludes before 100° C. with a mass loss of about 2.6%. Also provided in an embodiment is a process for the synthesis of crystalline Form J, as described below in Example 8, with ¹H NMR of crystalline Form J showing no residual solvent.

### Form L

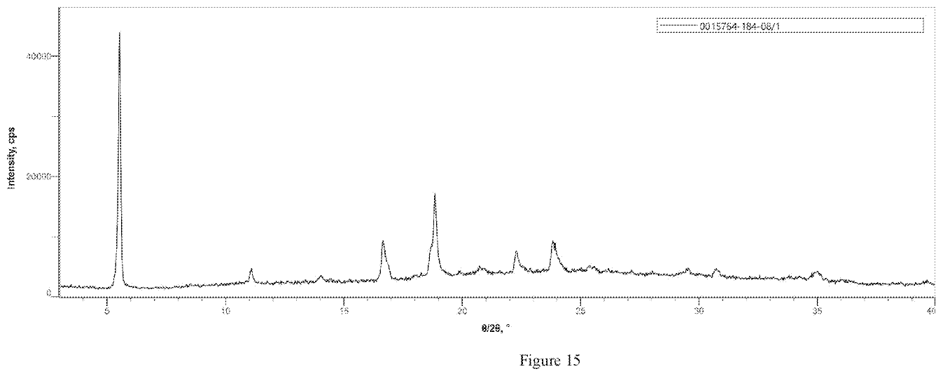

In additional embodiments is crystalline Form L of Compound 1, characterized by an X-ray powder diffractogram comprising signals as listed in Table 20 and as substantially shown in FIG. 15.

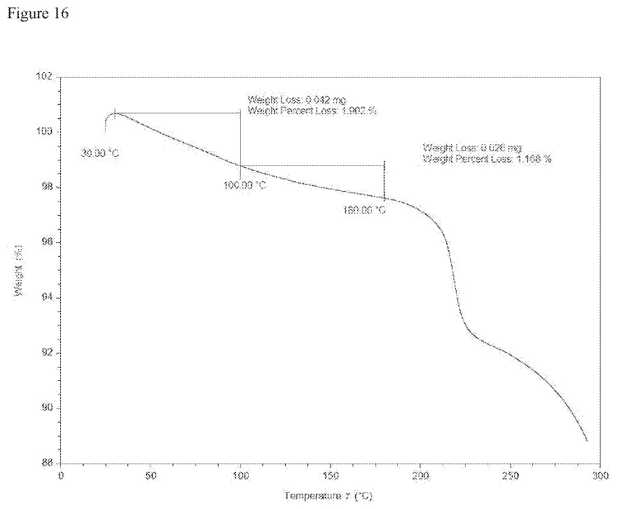

The TGA thermogram for Form L (FIG. 16) comprises a weight loss step that begins at about 30° C. and concludes before 100° C. with a mass loss of about 1.9%. The disclosure further provides in another embodiment a process for the synthesis of crystalline Form L, as described below in Example 9, with ¹H NMR of crystalline Form L showing less than 0.1% residual solvent.

### Form M

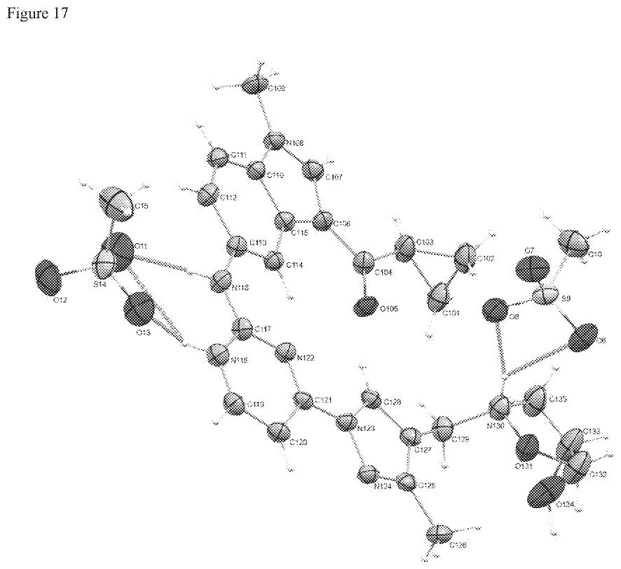

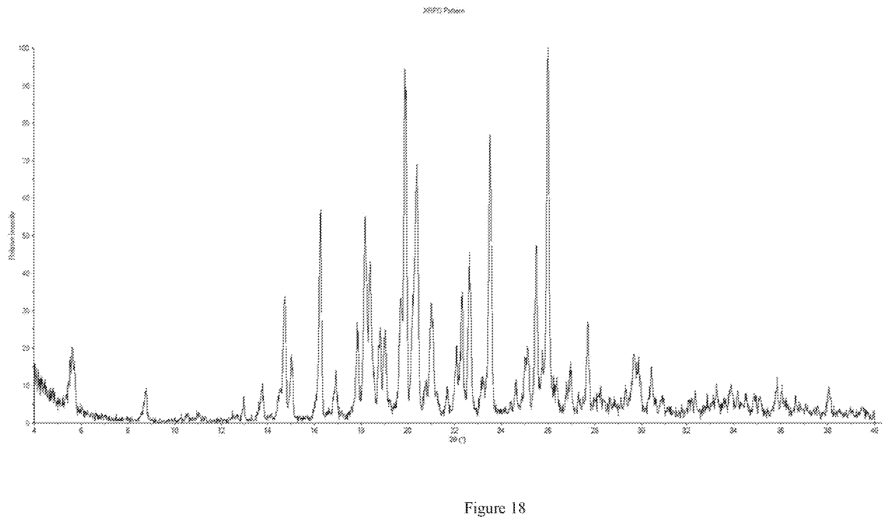

In one embodiment is crystalline Form M, which is an anhydrous form of Compound 1 (see Example 10). In an embodiment, Form M is characterized by single crystal X-ray diffraction (SCXRD; FIG. 17), wherein the unit cell has the following dimensions: a=9.603 Å, b=9.959 Å, c=15.765 Å, α=95.5°, β=93.29°, and γ=90.87° (see Table 21). In further embodiments, Form M is characterized by an XRPD pattern comprising signals as listed in Table 22 and a diffractogram substantially as shown in FIG. 18.

### Form N

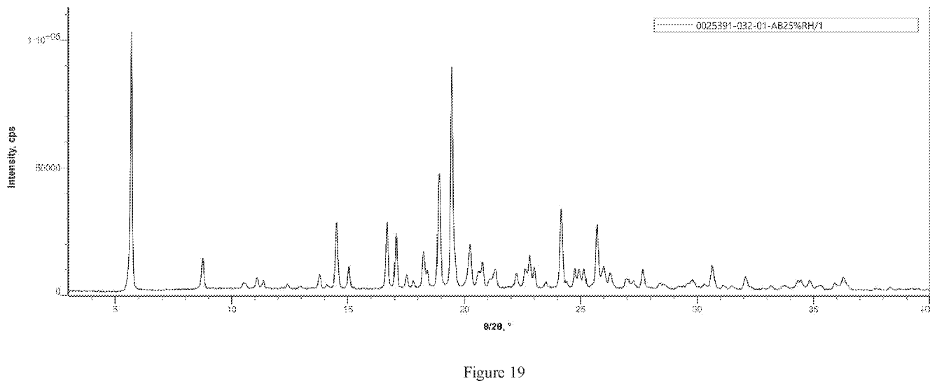

In another embodiment is crystalline Form N, a non-stoichiometric (i.e., partial) hydrate of Compound 1, characterized by an X-ray powder diffractogram comprising signals at 14.5, 16.7, 17.1, 19.4, 20.2, 24.1, and 25.7 2θ±0.2 °2θ as determined on a diffractometer using Cu-Kα radiation at a wavelength of 1.54 Å. In an embodiment, the XRPD pattern of Form N further comprises signals as listed in Table 23. In another embodiment, Form N is characterized by its X-ray powder diffractogram substantially as shown in FIG. 19.

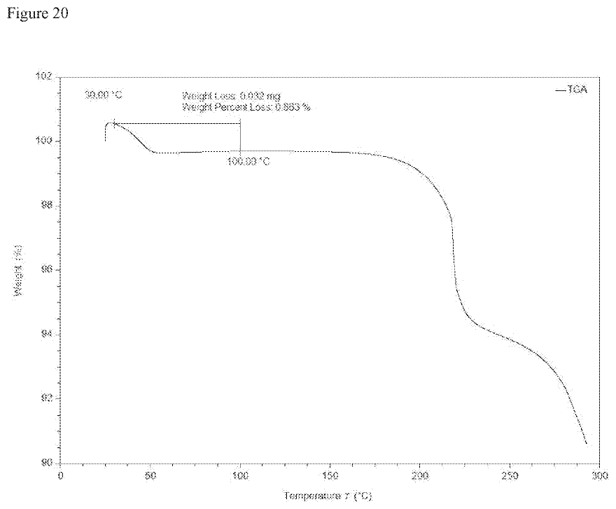

Form N is also characterized by a thermogravimetric analysis (TGA) thermogram comprising a weight loss of about 1% that begins at about 30° C. and concludes at about 100° C. In a further embodiment, Form N is characterized by a TGA thermogram substantially as shown in FIG. 20.

### Pharmaceutical Compositions

The disclosure also contemplates as another embodiment a pharmaceutical composition that comprises one or more crystalline forms of Compound 1 as described herein. As shown by the pharmacokinetic data herein, one advantage of the inventive crystalline forms, such as Form J, surprisingly resides in greater exposure. Therefore, a pharmaceutical composition can be formulated to contain a lower concentration of such a crystalline form.

In various embodiments, the therapeutically effective amounts of one or more crystalline forms in a pharmaceutical composition provide a dose of about 0.01 mg/kg to about 100 mg/kg per day. Typical dosages can vary from about 0.1 mg/kg to about 100 mg/kg per day, from about 0.1 mg/kg to about 1 mg/kg per day, from about 0.1 mg/kg to about 5 mg/kg per day, from about 0.1 mg/kg to about 10 mg/kg per day, from about 1 mg/kg to about 100 mg/kg per day, from about 10 mg/kg to about 100 mg/kg per day, from about 20 mg/kg to about 100 mg/kg per day, from about 30 mg/kg to about 100 mg/kg per day, from about 40 mg/kg to about 100 mg/kg per day, from about 50 mg/kg to about 100 mg/kg per day, from about 60 mg/kg to about 100 mg/kg per day, from about 70 mg/kg to about 100 mg/kg per day, from about 80 mg/kg to about 100 mg/kg per day, or from about 90 mg/kg to about 100 mg/kg per day. Intermediate dose ranges also are contemplated.

The pharmaceutical composition further comprises, in accordance with accepted practices of pharmaceutical compounding, one or more pharmaceutically acceptable excipients, diluents, adjuvants, stabilizers, emulsifiers, preservatives, colorants, buffers, or flavor imparting agents, that in aggregate constitute a pharmaceutically acceptable carrier. In general, the pharmaceutical composition is prepared with conventional materials and techniques, such as mixing, blending, and the like. In one embodiment the pharmaceutically acceptable carrier and, hence, the pharmaceutical compositions are solids.

Suitable pharmaceutically acceptable carriers, diluents, adjuvants, or excipients for use in the pharmaceutical compositions of the invention include tablets (coated tablets) made of, for example, collidone, shellac, gum Arabic, talc, titanium dioxide or sugar, capsules (gelatin or HPMC), syrups containing suspensions of the active substances, emulsions or inhalable powders (of various saccharides such as lactose or glucose, salts and mixture of these excipients with one another) and aerosols.

Other excipients which may be used include, for example, carriers such as natural mineral powders (e.g., kaoline, clays, talc, chalk), synthetic mineral powders (e.g., highly dispersed silicic acid and silicates), sugars (e.g., cane sugar, lactose, and glucose), emulsifiers (e.g., lignin, spent sulphite liquors, methylcellulose, starch and polyvinylpyrrolidone) and lubricants (e.g., magnesium stearate, talc, stearic acid and sodium lauryl sulphate).

In accordance with some embodiments, the pharmaceutical composition comprises one or more crystalline forms as disclosed herein. For example, the composition comprises two forms, three forms, or four forms. An exemplary composition comprises Form J and Form F or Form J and Form N. Binary compositions, i.e., those containing just two forms, provide for various weight ratios of these forms.

In one embodiment, the pharmaceutical composition comprises a therapeutically effective amount of Form J and (ii) solid pharmaceutically acceptable excipients. In one embodiment, the pharmaceutical composition comprises a therapeutically effective amount of Form J and Form F and (ii) solid pharmaceutically acceptable excipients. In one embodiment, the pharmaceutical composition comprises a therapeutically effective amount of Form J and Form N, and (ii) solid pharmaceutically acceptable excipients. In one embodiment, the pharmaceutical composition comprises a therapeutically effective amount of Form J, Form F, and Form N, and (ii) solid pharmaceutically acceptable excipients.

For tablet compositions, the inventive crystalline forms are combined with non-toxic pharmaceutically acceptable excipients for the manufacture of tablets.

The compositions may also include other inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated, or they may be coated by known coating techniques.

Carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone) may also be included in a composition.

### Methods of Use

The crystalline forms of the present disclosure were found to be inhibitors of SYK kinase activity and have therapeutic benefit in the treatment of disorders associated with inappropriate kinase activity, for example, in the treatment and prevention of disease states mediated by kinases, including SYK kinase. Therefore, the present invention provides methods of regulating and inhibiting signal transduction cascades in which a kinase plays a role. The method comprises administering to a subject or contacting a cell expressing the kinase with an effective amount of a crystalline form described herein, and/or a pharmaceutical composition of a crystalline form, to regulate or inhibit the signal transduction cascade. The methods are also used to regulate and, especially inhibit downstream processes or cellular responses elicited by activation of specific kinase signal transduction cascades.

In one embodiment, compositions of the inventive crystalline forms are used for the treatment of a protein kinase-mediated disease, disorder, or a condition mediated by inappropriate protein kinase activity. Examples of disease classes treated by therapeutics comprising the inventive crystal forms include without limitation immunological disorders, hematological disorders including cancers of the blood and bone marrow, rare genetic disorders, anemia, HIV related conditions, sickle cell anemia, as well as other complications that arise due to chemotherapy and transfusion.

In one embodiment, compositions of the inventive crystalline forms are used for the treatment of an autoimmune condition, the inflammatory diseases and/or allergic disorders such as conjunctivitis, allergy, allergic rhinoconjunctivitis, autoimmune bullous conditions including pemphigus and pemphigoid, mastocytosis, anaphylaxis, idiopathic thrombocytopenic purpura (ITP), Berger's disease, Evans syndrome, Guillain-Barre syndrome, granulocytopenia, Goodpasture's syndrome, hepatitis, Henoch-Schonlein purpura, warm autoimmune hemolytic anemia (wAIHA), immunohaemolytic anaemia, autoimmune haemolytic anemia, systemic lupus erythematosus (SLE), discoid cutaneous lupus, heparin-induced thrombocytopenia, antiphospholipid syndrome (APS), thrombotic thrombocytopenic purpura, asthma, severe asthma, chronic obstructive pulmonary disease (COPD), adult respiratory distress syndrome (ARDS), ulcerative colitis, Crohn's disease, bronchitis, dermatitis, allergic rhinitis, psoriasis, scleroderma (contact and allergic), pemphigus, chronic (spontaneous) urticaria, bullous disorders, collagenoses, contact dermatitis eczema, Kawasaki Disease, rosacea, Sjogren-Larsso Syndrome, Wegners granulomatosis and other vasculitides, idiopathic thrombocytopenia purpura, giant cell arteriosis, glomerulonephritis, chronic transplant rejection, adult respiratory distress syndrome, rheumatoid arthritis, multiple sclerosis, inflammatory bowel syndrome, HIV, diffuse large B cell lymphoma, Non-Hodgkin lymphoma, osteosarcoma, melanoma, breast cancer, renal cancer, prostate cancer, colorectal cancer, thyroid cancer, ovarian cancer, pancreatic cancer, neuronal cancer, lung cancer, uterine cancer, gastrointestinal cancer, Alzheimer's disease, Parkinson's disease, osteoporosis, osteopenia, osteomalacia, osteofibrosis, Paget's disease, diabetes mellitus, blood vessel proliferative disorders, ocular diseases, cardiovascular disease, restenosis, fibrosis, atherosclerosis, arrhythmia, angina, myocardial ischemia, myocardial infarction, cardiac or vascular aneurysm, vasculitis, stroke, peripheral obstructive arteriopathy, reperfusion injury following ischemia of an organ or a tissue, endotoxic, surgical or traumatic shock, hypertension, valvular heart disease, heart failure, abnormal blood pressure, vasoconstriction, vascular abnormality, transplant rejection, and infectious diseases including viral and fungal infections.

In one embodiment, compositions of the inventive crystalline forms are used for the treatment of a disease, or a disorder mediated by inappropriate activity of SYK kinase. Such SYK-mediated disease states include, but are not limited to, inflammatory, respiratory diseases and autoimmune diseases, such as, by way of example only, rheumatoid arthritis, systemic lupus erythematosus (SLE), asthma, chronic obstructive pulmonary disease (COPD), adult respiratory distress syndrome (ARDs), ulcerative colitis, Crohn's disease, allergic rhinitis, bronchitis, dermatitis, allergic rhinitis, psoriasis, scleroderma, urticaria, rheumatoid arthritis, multiple sclerosis, cancer, HIV-associated disease and non-Hodgkin lymphoma including diffuse large B cell lymphoma, follicular, mantle cell, capsule cell lymphoma, small lymphocytic lymphoma, chronic lymphocytic lymphoma, Burkitt diffuse large B cell lymphoma, and T cell lymphoma.

In one embodiment, compositions of the inventive crystalline forms are used for the treatment of idiopathic thrombocytopenic purpura (ITP), rheumatoid arthritis (RA), warm autoimmune hemolytic anemia (wAIHA), systemic lupus erythematosus, Psoriasis, and antiphospholipid syndrome (APS).

In the context of the inventive methods and uses, the “subject” to be treated with an inventive crystalline form is an animal, such as a mammal, e.g., human, dogs, cats, mice, monkeys, rats, rabbits, horses, cows, guinea pigs, sheep. In one embodiment, the subject is a human.

### EXAMPLES

The following non-limiting examples are further embodiments of, and are intended to illustrate, the present disclosure.

### Pharmacokinetic Study

Plasma pharmacokinetics (PK) of different crystalline forms of Compound 1 were investigated using male SD rats (N=3/group). Capsules comprising a specific crystalline form of SKI-2-703 were administered orally, according to the study design shown in Table 1 below:

TABLE 1

| | | Dose Level | | Fill | Administration | No. of |
|---|---|---|---|---|---|---|
| Group | Treatment | (mg/kg) | Capsule/Size | weight(mg) * | Route | Animals |
| 1 | Compound 1 | 35 | GASTRIC | 8.75 | Oral capsule | 3 Males |
| | (Form B) | | CAPSULE/ | | | |
| 2 | Compound 1 | 35 | SIZE 9 | 8.75 | Oral capsule | 3 Males |
| | (Form F) | | 1X | | | |
| 3 | Compound 1 | 35 | | 8.75 | Oral capsule | 3 Males |
| | (Form J) | | | | | |
| 4 | Compound 1 | 35 | | 8.75 | Oral capsule | 3 Males |
| | (Form N) | | | | | |
| 5 | Compound 1 | 35 | | 8.75 | Oral capsule | 3 Males |
| | (Amorphous) | | | | | |

* Based on a 250 g animal

Plasma samples were collected at 0.5 h, 1 h, 2 h, 4 h, 8 h, 12 and 24 h post-dose. The concentrations of SKI-O-592 were determined in plasma and a summary of the mean plasma PK parameters are provided in Table 2.

TABLE 2

| | | | % AUC_{(0-24 h)} | | | |
|---|---|---|---|---|---|---|
| | Compound 1 Form | AUC_{(0-24 h)} | versus | Cₘₐₓ | Tₘₐₓ | t_{1/2} |
| Group | administered | (h · ng/mL) | Amorphous | (ng/mL) | (h) | (h) |
| 1 | Form B | 5295 | 68% | 2568 | 0.83 | 0.89 |
| 2 | Form F | 6509 | 84% | 2870 | 0.67 | 0.92 |
| 3 | Form J | 8939 | 116% | 2950 | 1.83 | 0.92 |
| 4 | Form N | 7195 | 93% | 3910 | 0.67 | 0.87 |
| 5 | Amorphous | 7730 | 100% | 3787 | 1.67 | 0.91 |

These PK data show greater absorption and exposure (AUC_{0-24 h}) in male SD rats orally administered gastric capsules containing Compound 1 Form J when compared to amorphous SKI-O-703. Thus, one advantage of crystalline Form J of Compound 1 as a therapeutic for the treatment of diseases and conditions mentioned herein resides in a better benefit-risk profile.

### Solubility Studies

The approximate solubility of Form F of Compound 1 was determined by contacting a weighed sample with aliquots of the test solvent at room temperature. Complete dissolution of the test material was determined by visual inspection. Solubility was estimated based on the total solvent used to provide complete dissolution. The solubility at 25° C. and 50° C. for the various test solvents is shown in Table 3 and expressed as “less than” or as a range if dissolution did not occur during the experiment.

TABLE 3
Solubility of Form F

Solubility (mg/ml)

| Solvents | 25° C. | 50° C. |
|---|---|---|
| Acetone | S < 5.3 | S < 5.3 |
| MEK | S < 5.5 | S < 5.5 |
| THF | S < 6.1 | S < 6.1 |
| ACN | S < 6.1 | S < 6.1 |
| 2-MeTHF | S < 5.5 | S < 5.5 |
| H₂O | S > 220 | N/A |
| Heptane | S < 5.1 | S < 5.1 |
| DMSO | 73.3 < S < 110 | N/A |
| EtOH/H₂O = 95/5(v/v) | S < 8.46 | S > 8.46 |
| Acetone/H₂O = 95/5(v/v) | S < 6.1 | S < 6.1 |
| THF/H₂O = 95/5(v/v) | S < 6.1 | S < 6.1 |
| ACN/H₂O = 95/5(v/v) | S < 5.5 | S < 5.5 |
| Acetone/H₂O = 1/1(v/v) | 120 < S < 240 | N/A |
| Acetone/H₂O = 5/1(v/v) | 12.5 < S < 14.3 | N/A |
| 1,4-Dioxane | S < 5.5 | S < 5.5 |

N/A: Not Available

Table 4 summarizes the results of a study that measured the rate at which Form E and Form F dissolve in water, simulated gastric fluid (SGF), fasted simulated intestinal fluid (FaSSIF), and fed simulated intestinal fluid (FeSSIF) at 37° C.

XRPD analysis of these test mixtures at 1 hour, 4 hours, and 24 hours were also performed, (see Table 4), to evaluate changes in the physical states of Form E and Form F.

TABLE 4

| | | Solubility | | | | | | | | |
|---|---|---|---|---|---|---|---|---|---|---|
| | | (mg/ml) at 37° C. | | | | XRPD | | | | pH |
| Media | Material | 0.5 h | 1 h | 4 h | 24 h | 0.5 h | 1 h | 4 h | 24 h | 24 h |
| water | CPo130033-01- | 0.083 | 0.101 | 0.100 | 0.086 | Poor | Poor | Poor | Poor | 1.72 |
| | 03-04 | | | | | crystalline | crystalline | crystalline | crystalline | |
| SGF | Form F | S > 6.25 | S > 6.25 | S > 6.25 | S > 6.25 | N/A | N/A | N/A | N/A | 1.23 |
| FaSSIF | | 0.005 | 0.003 | 0.004 | 0.005 | Poor | Poor | Poor | Poor | 2.78 |
| | | | | | | crystalline | crystalline | crystalline | crystalline | |
| FeSSIF | | 0.008 | 0.007 | 0.007 | 0.008 | Poor | Poor | Poor | Poor | 4.73 |
| | | | | | | crystalline | crystalline | crystalline | crystalline | |
| water | 0025391-032-01 | 2.723 | 0.096 | 0.090 | 0.076 | Amorphous | Poor | Poor | Poor | 1.78 |
| | Form E | | | | | | crystalline | crystalline | crystalline | |
| SGF | | S > 6.25 | S > 6.25 | S > 6.25 | S > 6.25 | N/A | N/A | N/A | N/A | 0.93 |
| FaSSIF | | 0.006 | 0.004 | 0.005 | 0.004 | Poor | Poor | Poor | Little solid | 12.74 |
| | | | | | | crystalline | crystalline | crystalline | | |
| FeSSIF | | 0.008 | 0.008 | 0.007 | 0.008 | Poor | Poor | Poor | Poor | 4.76 |
| | | | | | | crystalline | crystalline | crystalline | crystalline | |

N/A: Not Available The data in Table 4 shows that while Form E and Form F dissolve in all the test solvents, the physical state of the crystalline forms of Form E and Form F changes/degrades rapidly, as evidenced by the XRPD data at 0.5 hours for all test solvents. Table 4 also shows that the solubilities of Form E and Form F are greatest in strong acidic media such as SGF, (S > 6.62 mg/ml).

### Relative Solution Phase Stability Studies—Forms E, F, and J of Compound 1

Stability studies were carried out using a 1:1 w/w ratio of Forms E and Form F as input materials. Stability was evaluated at different temperatures and in different solvents and solvent mixtures. XRPD was used to evaluate if the starting crystalline Form E and Form F were altered by the tested solvents and solvent mixture after 24 hours and 72 hours. The results from these studies are summarized in Table 5.

TABLE 5

| Solvent | Temperature | XRPD (1 day) | XRPD (3 day) |
|---|---|---|---|
| MeOH | 20-25° C. | Form E | Form E |
| EtOH | | Form E | Form E |
| 2-MeTHF | | Form E + Form F | Form E + |
| | | | Monomesylate Form D |
| Acetone | | Form J | Form J |
| Acetone/H2O 95/5 | | Form E + Form F | Form J |
| Acetone/H2O 95/5 | | Form J | Form J |
| Acetone/H2O 80/20 | | Form E + Form F | Form J + |
| | | | Monomesylate Form D |
| Acetone/H2O 1/1 | | Form J + | Form J + |
| | | Monomesylate Form D | Monomesylate Form D |
| EtOH/H2O 80/20 | | Form J + | Form J + |
| | | Monomesylate Form D | Monomesylate Form D |
| MeOH/H2O 95/5 | | Form J + | Form J + |
| | | Monomesylate Form D | Monomesylate Form D |
| Acetone | 0° C. | Form E + Form F | Form J |
| Acetone/H2O 95/5 | | Form J | Form J |
| Acetone/H2O 5/1 | | Form J + | Form J + |
| | | Monomesylate Form D | Monomesylate Form D |
| Acetone/H2O 1/1 | | Form J + | Form J + |
| | | Monomesylate Form D | Monomesylate Form D |
| Acetone | −10° C. | Form E + Form F | Form J |
| Acetone/H2O 95/5 | | Form E + Form F | Form J |
| Acetone/H2O 5/1 | | Form J + | Form J + |
| | | Monomesylate Form D | Monomesylate Form D |
| Acetone/H2O 1/1 | | Form J + | Form J + |
| | | Monomesylate Form D | Monomesylate Form D |

Table 5 also shows stability data for Form E and Form F in acetone and acetone-water mixtures at three temperatures, specifically −10° C., 0° C., and 25° C. XR-PD analysis of the solid from these competitive ripening studies at 24 hours and 72 hours showed that Form J is the more stable crystal form. It is evident from the data in Table 5 that some disproportionation occurs during crystallization in many of the tested solvent systems, particularly those with higher water content, including the acetone/water mixed solvent system.

Furthermore, the form stabilities of a 1:1 mixture of Form F and Form J in (a) DMSO/acetone/water (8V/OV/1.2V) at 50° C. and in (b) DMSO/acetone/water (8V/16V/1.2V) at 20° C. was evaluated over 6 days using XRPD.

Table 6 shows the results from this time course study. For the DMSO/acetone/water (8V/OV/1.2V) solvent system at 50° C., complete conversion of the 1:1 mixture of Form F and Form J to Form J occurred within 3 hours.

However, when the composition of the solvent mixture used is changed to DMSO/acetone/water (8V/16V/1.2V; 20° C.), XRPD data showed a mixture of Form J and Form F over 48 hours (2 days), with complete conversion of the mixture of Form F and Form J to a single crystal form, namely, Form J after incubation for 4 days. These data clearly illustrate the impact of solvent on the physical stability of the crystalline forms, and the data further evidence Form J as more stable than Form F.

TABLE 6

| | | XRPD | | | | | | | |
|---|---|---|---|---|---|---|---|---|---|
| Starting | Solvent// | T0 | T1 | T2 | T3 | T4 | T5 | T6 (4 | T7 (6 |
| Material | Temp. | (10 min) | (1 h) | (3 h) | (6 h) | (24 h) | (48 h) | days) | days) |
| Form F + | DMSO/acetone/water | Form J + | Form J + | Form | Form | N/A | N/A | N/A | N/A |
| Form J | 8 V/0 V/1.2 V // | F | F | J | J | | | | |
| (1:1) | 50° C. | | | | | | | | |
| Form F + | DMSO/acetone/water | Form J + | Form J + | Form J + | Form J + | Form J + | Form J + | Form J | Form J |
| Form J | 8 V/16 V/1.2 V // | F | F | F | F | F | F | | |
| (1:1) | 20° C. | | | | | | | | |

N/A: Not Available

### Solid State Stability of Form E and Form F

The form stability of 2 test samples, Form E and Form F, respectively, were investigated under stress conditions, that is, upon exposure to light (4500 Lux, open pan), high temperature (60° C., closed pan), and high relative humidity (90±5%, open pan at 25° C.). XRPD analysis was used to monitor changes to the physical form and chemical stability of Form E and Form F when exposed to these conditions at 5-days and at 11-days. The data from these experiments are summarized in Table 7.

TABLE 7

| Lot | | Day 0 | | Day 5 | | Day 11 | |
|---|---|---|---|---|---|---|---|
| Number | Stress Condition | XRPD | Purity | XRPD | Purity | XRPD | Purity |
| 0025391- | Photo 4500Lux, | Form E | N/A | Form J | 99.56% | Form J | 99.46% |
| 013-04 | open pan | | | | | | |
| | 60° C., closed pan | | | Form E | 99.70% | Form J | 99.68% |
| | 25° C., RH90 ± 5%, | | | Form J | 99.80% | From J | 99.73% |
| | open pan | | | | | | |
| CPo130033- | Photo 4500Lux, | Form F | 99.7% | Form F | 99.79% | Form F | 99.62% |
| 01-03-04 | open pan | | | | | | |
| | 60° C., closed pan | | | Form F | 99.88% | Form F | 99.74% |
| | 25° C., RH90 ± 5%, | | | Form F | 99.90% | Form F | 99.78% |
| | open pan | | | | | | |

N/A: Not Available

XRPD data in Table 7 show that the sample of Form E converts to Form J when exposed to high relative humidity, high temperature, or intense light over 11-days. Over a 5-day exposure to light and high RH, Form E converts to Form J, while no changes in physical form or stability were observed for Form E test sample exposed to high temperatures (60° C.), over a 5-day period. In contrast, no change in the physical form and stability of Form F were observed when exposed to light, high temperature, and/or high relative humidity over the 11-day exposure period. Additionally, no changes in color were visually observed for Form E and Form F test samples exposed to light, high temperature, or high RH. Taken together, these data suggest that Form F is stable under the experimentally tested stress conditions. Form E, however, converts to Form J when exposed to light and high RH over a 5-day exposure, with no further changes in the physical state of Form J on longer exposure to the experimentally tested stress conditions.

### Interconversion of Crystal Forms:

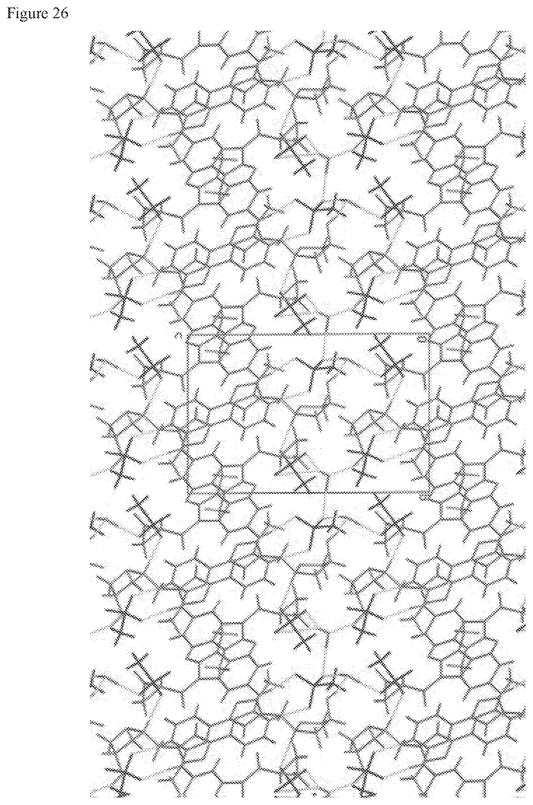

Crystalline Form E is a partial hydrate while crystalline Form J is a monohydrate of Compound 1. The present inventors found that Form E can be converted to Form J by exposing solid Form E to conditions of high relative humidity, e.g., RH>65%. FIG. 26 provides an explanation for the monohydrate nature of crystal Form J. FIG. 26 shows crystal packing within a unit cell of Form J. As seen from this figure, a channel-like structure is observed along the [1,0,0]direction of the unit cell. Without wishing to be bound by any particular theory, the present inventors hypothesize that this channel-like structure most likely allows easier water movement from the crystalline structure, thus accounting for conversion between Form J, Form E, Form N, and Form M when the relative humidity is changed.

Conversely, Form J (monohydrate) can be converted to Form E (partial hydrate) by drying the sample at a temperature in the range from about 50° C.-about 60° C. under vacuum. Similarly, Form M, an anhydrate, converts to Form J when exposed to an atmosphere of high RH (RH>65%) over a period of 6 hours, while exposure of the anhydrate Form M to relative humidity of ~20% resulted in the formation of a partial hydrate, Form N. Table 8 shows crystallographic data, such as the unit cell dimensions for Form M (anhydrate), Form N (partial hydrate), and Form J (monohydrate).

TABLE 8

| Polymorphic form | Monohydrate Form J | Partial hydrate Form N | Anhydrate Form M |
|---|---|---|---|
| Empirical formula | C₂₅H₂₉N₇O₃⁺² 2CH₃SO₃_{^{−}} | C₂₅H₂₉N₇O₃⁺² 2CH₃SO₃_{^{−}} | C₂₅H₂₉N₇O₃⁺² 2CH₃SO₃_{^{−}} |
| | H₂O | nH₂O | |
| Formula weight | 683.75 | | 665.74 |
| T [K] | 100(2) | 296(2) | 296(2) |
| λ [Å] | 1.54178 | 1.54178 | 1.54178 |
| Crystal system | Triclinic | Triclinic | Triclinic |
| Space group | P1 | P1 | P1 |

Unit cell dimensions

| | | | |
|---|---|---|---|
| a [Å] | 9.5479(8) | 9.6976(4) | 9.6850(5) |
| b [Å] | 10.2586(9) | 10.1696(6) | 10.0082(5) |
| c [Å] | 15.6585(13) | 15.6682(8) | 15.7940(8) |
| α [°] | 89.580(5) | 91.771(3) | 95.121(3) |
| β [°] | 88.352(5) | 93.419(2) | 93.784(3) |
| χ [°] | 87.868(5) | 92.421(2) | 91.375(3) |
| V[Å³] | 1532.0(2) | 1540.2(2) | 1520.78(13) |
| Z | 2 | 2(2) | 2 |
| Dc [g/cm3] | 1.482 | 1.436 | 1.454 |

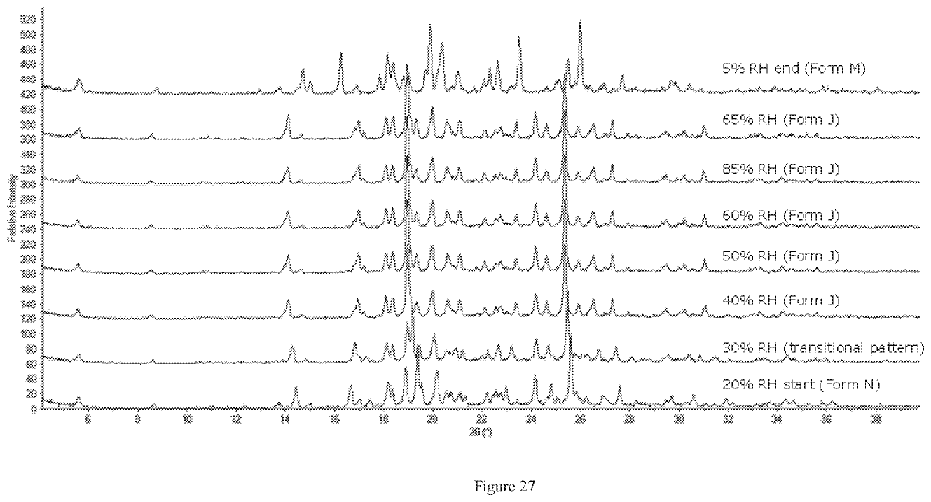

FIG. 27 shows Variable Humidity XRPD of Compound 1. Crystal Form N was used as the starting solid form for this study. The XRPD of Form N, a partial hydrate, was measured first at 20% RH. The RH was then increased in increments of 10% from an initial level of 20% RH to 30%, 40%, 50%, and 60% RH. As shown in FIG. 27, at a relative humidity of 20%, XRPD data supports crystalline Form N. Increasing the humidity to 30% RH gave an X-ray powder diffractogram of mixture of Form N and Form J with unassignable peaks to Form N and Form J. Further increases in relative humidity to 40%, 50%, and 60% gave XRPD diffractograms for crystal Form J. Raising the relative humidity of Form J solid to 85% did not change the form. However, the solid became deliquescent when the relative humidity is greater than 85%. XRPD analysis further showed that the solid continues to be a Form J crystalline material even when RH is decreased from 85% to 65% RH. However, when RH is further lowered to 5%, XRPD diffractogram showed signals that conform to anhydrous solid Form M.

### Dynamic Vapor Sorption (DVS)—Forms J, M, and N

Dynamic vapor sorption (DVS) is a gravimetric technique used to measure how quickly and how much vapor, typically water, is absorbed or adsorbed by a sample. This technique involves varying the vapor concentration around the sample and measuring the resulting change in mass. In a DVS analysis, the sample is placed on an ultra-sensitive balance in a controlled environment where the humidity is gradually increased or decreased. The change in mass of the sample as it absorbs or desorbs vapor is recorded. This process can be repeated over a range of relative humidity levels to generate sorption isotherms, which describe the equilibrium amount of vapor absorbed as a function of relative humidity at a constant temperature.

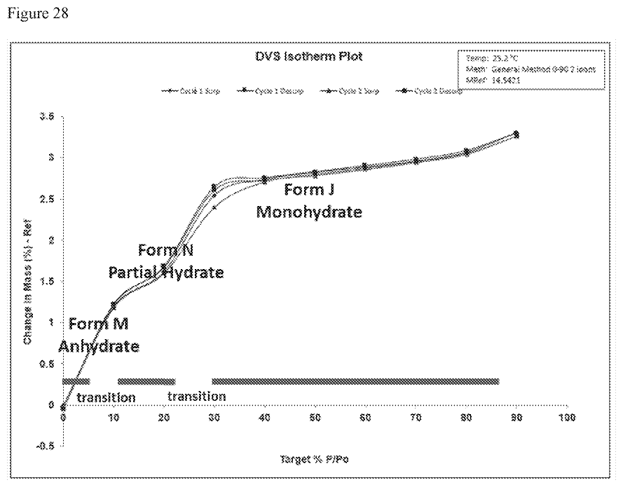

An exemplary DVS for Form J (monohydrate), Form M (anhydrate) and Form N (partial hydrate) of Compound 1 is shown in FIG. 28. Form M exhibits significant hygroscopicity taking up about 1.3 wt. % water vapor between 0% and 10% RH. Between 10% and 20% RH, the compound absorbs about 0.3 wt. % water vapor indicating a crystalline form that is partially hydrated, presumably a hemihydrate (Form N). The solid absorbs about 1.1 wt. % water vapor when the relative humidity is further increased to about 30%. Any further increases in relative humidity do not increase in the mass of the solid up to a relative humidity of about 85%, indicating a solid form that has absorbed the maximum amount of water vapor. XRPD of this solid gave signals corresponding to Form J (monohydrate). The desorption of water followed a trend similar to sorption, namely, no change in mass when RH is decrease from 85% to about 30%, followed by a loss of about 1.1 wt. % when RH is decreased from 30% to 20% and a final decrease in weight of 1.3% when RH is decreased from about 10% to 0%, indicating that the solid has lost its water molecule, and the material was an anhydrate at the start of the analysis.

### SYNTHESIS EXAMPLES

### Synthesis of Amorphous Compound 1

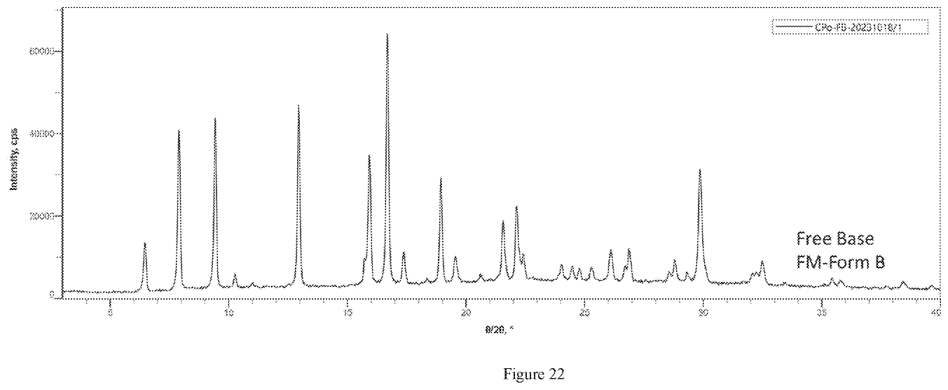

Amorphous Compound 1 was synthesized by contacting SKI-O-592 (2.0 g) with methane sulfonic acid (MSA, 2.05 eq.), using water (10.0 vol.) as the solvent. The clear solution thus obtained was frozen solid using an ethanol/dry ice mixture for ~3.0 hours and then lyophilized to dryness. XRPD of the lyophilized solid showed an amorphous powder (FIG. 22).

Alternatively, Form N (15 g) was contacted with water (10 vol.). The resultant mixture was stirred to dissolve the solid and then filtered. The filtrate was frozen and lyophilized to give amorphous Compound 1.

### General Protocol for Salt Formation

Compound 1 was prepared using the free base cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone (SKI-O-592). SKI-O-592 (50 mg) was contacted with a test solvent (1 mL) and the mixture was stirred at room temperature (~20° C.), to obtain a clear solution or suspension. This mixture was then contacted with a solution of methane sulfonic acid (MSA, 2.5 eq.), in the same test solvent.

The resultant mixture was heated and stirred at 50° C. for 2 h, followed by gradual cooling (5° C./h) until the temperature of the reaction mixture was 25° C. (room temperature). The room temperature mixture was stirred overnight to induce precipitation. The precipitate formed was centrifuged and the solid vacuum dried at 25° C. overnight. Alternatively, the wet solid is vacuum dried at 50° C. for 24 hours and the dry solid obtained was characterized by XRPD, DSC, TGA, and ¹H-NMR.

### General Protocol for Polymorph Screening

Once solid samples were harvested from crystallization experiments, they were either examined under a microscope for morphology or observed with the naked eye. Any crystalline shape was noted, but sometimes the solid exhibited unknown morphology, due to small particle size. Solid samples were then analyzed by XRPD, and the patterns were compared to each other to identify new crystalline or non-crystalline forms.

### 1. Slurry Ripening at 25° C.

16 vials comprising 30 mg of Compound 1 per vial were contacted with test solvents (2 ml/vial). The suspensions were stirred at 25±0.5° C. for 72 hours. The appearance of each vial was checked regularly. Additional Compound 1 was added if a clear solution was obtained upon stirring. Test amounts of the solids that precipitated out in individual vials were collected at days 3 and 14 and analyzed by XRPD.

### 2. Slurry Ripening at 50° C.

30 mg of a Compound 1 was added to 16 glass vials followed by the addition of corresponding test solvents (2 ml), to obtain suspensions. The samples were stirred at 50±0.5° C. for 72 hours. The appearance of each vial was checked regularly, and additional Compound 1 was added if a clear solution was obtained upon stirring. Test amounts of the solids that precipitated out in individual vials were collected at days 3, 5, or 14 and analyzed by XRPD.

### 3. Slow Evaporation at 25° C.

The glass vials comprising the filtrate from the slurry ripening study at 25° C., mentioned above, were maintained at room temperature (RT, 20~25° C.). The filtrate in each vial was blanketed under N₂ and each vial was sealed using a sealing film. A needle was used to puncture the sealing film of each vial and the vials were then placed in a fume hood to permit slow evaporation of the solvent. The solids from each vial were collected prior to complete dryness and examined by XRPD.

### 4. Fast Evaporation at 50° C.

The glass vials comprising the filtrate from the slurry ripening study at 50° C., mentioned above, were sealed with a sealing film, and then placed in an electric thermostatic drying oven maintained at 50° C. A needle was used to puncture the sealing film of each vial and the solvents were evaporated spontaneously. The solids samples from each vial were collected and examined by XRPD.

### 5. Precipitation Using a Solvent—Anti-Solvent System:

Several different solvent combinations were investigated. 30 mg of Compound 1 was dissolved in solvent in which the salt is highly soluble to obtain clear solution at 25° C. An anti-solvent was added to the clear solution dropwise with stirring until the solution becomes cloudy. Stirring was continued overnight to complete precipitation of solid. After stirring, the solid precipitate is filtered and the solids samples for each solvent—anti-solvent combination are collected and analyzed by XPRD.

### 6. Instrumentation

a. Powder X-Ray Diffraction (PXRD)

Throughout this disclosure are figures showing X-ray diffraction patterns and tables with signal lists. Signals within the range of up to about 40° 2θ were selected. Rounding algorithms were used to round each signal to the nearest 0.02° 2θ. The location of the signals along the x-axis (° 20) in both the figures and the lists were determined using Rigaku Smart Lab Studio II software and rounded to two significant figures after the decimal point.

XRPD was carried out using Rigaku Smartlab SE, multi-purpose diffractometer at a wavelength Cu, Kα, Kα1(Å):1.540598, Kα2(Å):1.544426, and Kα2:Kα1 intensity ratio:0.50. The X-Ray Tube setting was 40 kV, 15 mA, and the Scan Mode was 1 D. The scan speed (2 Theta) 10°/min.

XRPD signals are used to differentiate crystalline forms. Characteristic signals were determined by evaluating which representative signals, if any, are present in one crystalline form of a compound against all other known crystalline forms of that compound to within ±0.2° 2θ. Not all crystalline forms of a compound necessarily have at least one characteristic signal.

b. Differential Scanning Calorimetry (DSC): DSC was performed using a TA 2500 differential scanning calorimeter instrument. Temperature calibration was performed by measuring the melting point and the heat of melting against reported values. A sample was placed into a punched aluminum DSC pan, and the weight was accurately recorded. A weighed aluminum pan configured as the sample pan was placed on the reference side of the cell. The sample was heated under a nitrogen purge from ambient temperature (room temperature) to 300° C. at a heating rate of 10° C./min.

c. Thermogravimetry (TGA): TG analyses were performed using a TA, TGA500 instrument. TGA calibration was performed testing Curie points, namely, the magnetic transition of nickel (standard) from ferromagnetic to paramagnetic material prior to analysis of the samples. Each sample was placed in an open aluminum pan and inserted into the TG furnace. The furnace was heated under a nitrogen purge from ambient temperature (room temperature) to 300° C. at a heating rate of 10° C./min.

d. Polarized Light Microscopy: Samples were observed using an Olympus BX53 microscope. The sample was dispersed with methyl silicone oil on a glass slide and observed by PLM.

e. ¹HNMR Spectroscopy: Solution ¹H NMR spectra were acquired at 25° C. with a Bruker Avance Neo 400 MHz, DMSO-d6, 16 acquisition, relaxation delay (D1) of 1.0 sec. The samples were dissolved in an appropriate solvent such as D₂O or DMSO-d6. The residual peak from incompletely deuterated DMSO is at approximately 2.5 ppm, and that for incompletely deuterated water is 4.79 ppm.

f. High Pressure Liquid Chromatography (HPLC): HPLC analysis was carried out using an Agilent PA-A-LC-53 instrument. The column used was a Waters Sunfire C18+ (150 mm*4.6 mm*3.5 m) PN186002554, maintained at a temperature of 35° C. The flow rate was 1 mL/min., and the total run time was 33 minutes. The wavelength of detection was 210 nm, and the injection volume was 5 μl. Solvent gradient parameters are listed in the Table 9.

TABLE 9
Mobile Phase A: 10 Mm NH₄HCO₃ in Water
Mobile Phase B: ACN

| Time | % A | % B |
|---|---|---|
| 0.00 | 75 | 25 |
| 2.00 | 75 | 25 |
| 17.00 | 35 | 65 |
| 24.00 | 10 | 90 |
| 27.00 | 10 | 90 |
| 27.10 | 75 | 25 |
| 33.0 | 75 | 25 |

### Synthesis of Crystalline Forms

Described below are methodologies for the preparation of different crystalline forms of Compound 1. A summary of the physicochemical properties of the different forms of Compound 1 synthesized by the inventors is shown in Table 10.

TABLE 10

| | # of cases | | | |
|---|---|---|---|---|
| | observed | | % wt. loss | |
| | during | Crystallinity | at 100° C. | |
| Form | screening | by XRPD | by TGA | Remarks |
| A | 1 | High | 1.38% | Appear to be non-solvated; a single sample obtained |
| | | | | from salt formation study, not observed in polymorph |
| | | | | screen experiments. |
| B | 9 | Poor | 2.52% | Relatively poor crystallinity: all nine samples obtained |
| | | | | from salt formation. |
| C | 1 | Moderate | 6.21% | Moderate crystallinity: one sample obtained from salt |
| | | | | formation only; difficult to reproduce. |
| E | 51 | High | 1.80% | Dominant form from polymorph screening; XRPD |
| | | | | pattern is very similar to that of Form J; DVS curves are |
| | | | | essentially identical; can be treated as one form with |
| | | | | subtle structural variations/distortion for hydrates. |
| F | 40 | High | 2.50% | Monohydrate; 60 kg GMP batch; many samples obtained |
| | | | | from slurry ripening using Form F as the input; |
| | | | | thermodynamically less stable than Forms E and J; more |
| | | | | difficult to produce phase-pure Form F. |
| H | 5 | Moderate | 2.06% | Metastable form obtained from fast evaporation |
| | | | | experiments. |
| J | 28 | High | 2.75% | Monohydrate; stable in aqueous mixtures at high water |
| | | | | activities and in solid-state at high RH. See comments |
| | | | | for Form E above. |
| L | 1 | Poor | N/A | Poorly crystalline; obtained from anti-solvent addition in |
| | | | | DMSO/toluene. |
| M | 0 | High | N/A | Anhydrate; discovered initially from the calculated |
| | | | | XRPD pattern from single crystal structure data; stable |
| | | | | at <10% RH and dry N₂. |
| N | 0 | High | 0.86% | Non-stoichiometric; discovered initially during the |
| | | Crystallinity | | crystallization process development; partially |
| | | | | dehydrated form of Form E/J at 10-30% RH and RT. |

N/A: Not Available

### Example 1: Synthesis of Form A of Compound 1

Cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone (SKI-O-592) (50 mg) was dissolved in 1 mL acetone in a glass vial (2 ml). The mixture was stirred to obtain a suspension. A solution of methane sulfonic acid (MSA, 2.5 eq.) in acetone (0.5 ml) was separately prepared and added dropwise to the suspension of SKI-O-592. The resultant mixture was heated to 50° C. and stirred for 2 h. The heated mixture was then gradually cooled (5° C./h) to 25° C. and stirred overnight (10-15 h). The solid formed was collected by filtration and dried at RT (20-25° C.) under vacuum overnight (10-15 h), analyzed by XRPD to give a diffractogram shown in FIG. 1. The observed 2θ±0.2° 2θ signals for Form A of Compound 1 are shown in Table 11.

TABLE 11

| Signal No. | 2Theta(θ)° | Signal Intensity | Signal Intensity %. |
|---|---|---|---|
| 1 | 7.891 | 1943 | 4.79 |
| 2 | 8.444 | 1453 | 3.58 |
| 3 | 9.903 | 546 | 1.91 |
| 4 | 10.546 | 1079 | 6.72 |
| 5 | 10.962 | 1095 | 2.94 |
| 6 | 12.11 | 575 | 0.97 |
| 7 | 13.373 | 582 | 1.43 |
| 8 | 13.920 | 6145 | 22.89 |
| 9 | 14.347 | 1389 | 4.41 |
| 10 | 15.965 | 2558 | 7.30 |
| 11 | 16.527 | 3531 | 12.50 |
| 12 | 16.732 | 5912 | 13.04 |
| 13 | 16.987 | 932 | 2.81 |
| 14 | 17.633 | 1096 | 2.16 |
| 15 | 17.890 | 3118 | 10.48 |
| 16 | 18.233 | 2595 | 7.09 |
| 17 | 18.738 | 18961 | 100.00 |
| 18 | 19.758 | 4059 | 11.32 |
| 19 | 20.572 | 3176 | 21.39 |
| 20 | 22.146 | 2647 | 7.90 |
| 21 | 22.730 | 1511 | 8.29 |
| 22 | 23.246 | 1457 | 2.91 |
| 23 | 23.729 | 4316 | 12.48 |
| 24 | 24.012 | 1826 | 9.58 |
| 25 | 24.561 | 1443 | 2.83 |
| 26 | 25.188 | 5540 | 12.00 |
| 27 | 25.365 | 2383 | 14.95 |
| 28 | 25.77 | 456 | 0.78 |
| 29 | 26.077 | 1528 | 5.65 |
| 30 | 26.390 | 1650 | 5.12 |
| 31 | 27.003 | 2166 | 5.11 |
| 32 | 29.20 | 1208 | 12.78 |
| 33 | 29.831 | 1280 | 5.67 |
| 34 | 30.580 | 2367 | 8.97 |
| 35 | 30.927 | 828 | 2.21 |
| 36 | 32.71 | 409 | 0.62 |
| 37 | 32.98 | 525 | 2.11 |
| 38 | 33.14 | 495 | 0.49 |
| 39 | 34.08 | 1140 | 5.63 |
| 40 | 34.79 | 803 | 7.68 |
| 41 | 38.40 | 594 | 1.66 |
| 42 | 39.467 | 720 | 4.57 |

### Example 2: Preparation of Form B of SKI-O-592

Form J (110 g; Example 8) was contacted with an aqueous solution of sodium hydroxide (1M, 10 vol.). After stirring at room temperature, the wet solid was washed with water and slurred in ethanol (20 vol.), at room temperature for 5 days. After filtration, the solid obtained was dried and characterized by HPLC and XRPD. The results of these analysis show a characteristic XRPD diffractogram (FIG. 22), Form B of SKI-O-592. The HPLC purity of Form B free base was 94.7%.

### Example 3: Preparation of Form B of Compound 1

SKI-O-592 (50 mg) was dissolved in the test solvents (1 mL) shown in Table 12 below, and the mixture was stirred at room temperature (~20° C.), to obtain a clear solution or suspension. This mixture was then contacted with a solution of methane sulfonic acid (MSA, 2.5 eq.), in the same test solvent. The resultant mixture was heated and stirred at 50° C. for 2 h, followed by gradual cooling (5° C./h) to 25° C. The room temperature mixture was stirred overnight to induce precipitation. The precipitate formed was centrifuged and the solid vacuum dried. The dry solid was characterized by XRPD.

TABLE 12

| Starting | | |
|---|---|---|
| Material | Solvent | Results |
| SKI-O-592 | Methyl ethyl ketone | Poor crystallinity |
| SKI-O-592 | MIBK | Poor crystallinity |
| SKI-O-592 | THF | Poor crystallinity |
| SKI-O-592 | 2-Me-THF | Poor crystallinity |
| SKI-O-592 | ACN | Poor crystallinity |
| SKI-O-592 | MEK/water (95/5, v/v) | Form F + Form B |
| SKI-O-592 | ACN/water (95/5, v/v) | Form F + Form B |

XRPD data showed the solid product to be a mixture of Form F and Form B when MEK/water (95/5, v/v) and ACN/water (95/5, v/v) were used as solvents. XRPD analysis of the solid material from experiments using MEK, MIBK, THF, 2Me-THF, and ACN as solvents show solids of poor crystallinity.

Starting with Form J

Form J (10 g; Example 8) was dissolved in water (10 vol.). The resultant solution was freeze dried using a mixture of dry ice/ethanol and lyophilized to give an amorphous powder. This powder was then slurried in ethanol (10 vol.), at room temperature for 5 days. After filtration, the solid obtained was dried under vacuum and characterized by HPLC and XRPD. The results of these analysis show a XRPD diffractogram (FIG. 3), characteristic for Form B. The HPLC purity of this material was 99.6%. The observed 2θ±0.2° 2θ signals for Form B are shown in Table 13.

TABLE 13

| Signal | | Signal | Signal |
|---|---|---|---|
| No. | 2Theta(θ)° | Intensity | Intensity %. |
| 1 | 5.520 | 5354 | 49.18 |
| 2 | 7.994 | 1076 | 6.48 |
| 3 | 8.546 | 799 | 6.2 |
| 4 | 8.91 | 534 | 8.02 |
| 5 | 10.71 | 285 | 7.28 |
| 6 | 12.77 | 503 | 9.34 |
| 7 | 13.477 | 703 | 12.9 |
| 8 | 13.995 | 3059 | 52.54 |
| 9 | 16.74 | 1652 | 29.37 |
| 10 | 17.987 | 1603 | 15.21 |
| 11 | 18.345 | 2102 | 31.35 |
| 12 | 18.822 | 5394 | 74.61 |
| 13 | 19.183 | 2035 | 22.68 |
| 14 | 20.009 | 2304 | 41.84 |
| 15 | 20.628 | 2025 | 44.16 |
| 16 | 22.23 | 1461 | 7.62 |
| 17 | 23.80 | 1442 | 12.7 |
| 18 | 25.347 | 4167 | 100 |
| 19 | 27.095 | 1115 | 6.29 |
| 20 | 29.97 | 640 | 13.13 |
| 21 | 30.67 | 838 | 7.54 |
| 22 | 34.249 | 597 | 13.34 |

Starting with Amorphous Compound 1

Form B was also synthesized using a slurry ripening procedure. Amorphous SKI-O-703 (1.0 g) was contacted with ethanol (10 vol.) to obtain a suspension which was stirred at room temperature for 5 days. The suspension was then allowed to settle, centrifuged, and the solid material collected and dried. The XRPD analysis showed a diffractogram (FIG. 3), characteristic for Form B.

### Example 4: Preparation of Form C of Compound 1

The synthesis was carried out by dissolving SKI-O-592 (50 mg) in 1 mL 1,4-dioxane in a 2 ml glass vial. The mixture was stirred to obtain a suspension. A solution of methane sulfonic acid (MSA, 2.5 eq.) in 1,4-dioxane (0.5 ml) was separately prepared and added dropwise to the suspension of SKI-O-592. The resultant mixture was heated to 50° C. and stirred for 2 h. The heated mixture was then gradually cooled (5° C./h) to 25° C. and stirred overnight (10-15 h). The solid formed was collected by filtration and dried at RT (20-25° C.) under vacuum overnight (10-15 h). XRPD analysis showed a solid with low crystallinity, See FIG. 5. The observed 2θ±0.2 °2θ signals for Form C are shown in Table 14.

TABLE 14

| Signal | | Signal | Signal |
|---|---|---|---|
| No. | 2Theta(θ)° | Intensity | Intensity %. |
| 1 | 7.918 | 1070 | 6.62 |
| 2 | 8.441 | 549 | 10.15 |
| 3 | 10.43 | 289 | 13.05 |
| 4 | 10.95 | 262 | 1.86 |
| 5 | 13.33 | 408 | 6.06 |
| 6 | 13.913 | 1766 | 31.01 |
| 7 | 15.43 | 647 | 6.28 |
| 8 | 16.729 | 1400 | 26.43 |
| 9 | 18.19 | 1262 | 52.19 |
| 10 | 18.752 | 4069 | 100 |
| 11 | 19.775 | 1759 | 18.1 |
| 12 | 20.14 | 652 | 12.15 |
| 13 | 20.564 | 1400 | 23.49 |
| 14 | 22.153 | 806 | 5.93 |
| 15 | 23.70 | 779 | 27.52 |
| 16 | 25.209 | 2523 | 38.94 |
| 17 | 34.22 | 275 | 17.69 |

### Example 5: Preparation of Form E of Compound 1

Form E was obtained by charging 30 mg of Form F (Example 6) into a glass vial and contacting the material with methanol (0.3 ml), to obtain a solution. The solution was stirred at room temperature for 3 days, then filtered and the solid collected was dried prior to analysis. Table 15 shows the XRPD 2θ±0.2 °2θ signals for Form E and the complete X-ray powder diffractogram is shown in FIG. 7.

TABLE 15

| Signal | | Signal | Signal |
|---|---|---|---|
| No. | 2Theta(θ)° | Intensity | Intensity %. |
| 1 | 5.561 | 7706 | 49.17 |
| 2 | 8.575 | 4488 | 24.34 |
| 3 | 10.730 | 1370 | 11.45 |
| 4 | 11.096 | 546 | 3.79 |
| 5 | 12.30 | 883 | 4.05 |
| 6 | 13.461 | 980 | 3.48 |
| 7 | 14.0360 | 10801 | 68.27 |
| 8 | 14.455 | 2033 | 9.59 |
| 9 | 16.663 | 2462 | 21.6 |
| 10 | 16.886 | 7446 | 34.66 |
| 11 | 17.185 | 2290 | 11.15 |
| 12 | 18.010 | 8003 | 42.8 |
| 13 | 18.389 | 5182 | 27.34 |
| 14 | 18.677 | 6654 | 37.03 |
| 15 | 18.852 | 21736 | 100 |
| 16 | 18.980 | 3752 | 26.04 |
| 17 | 19.230 | 4232 | 35.72 |
| 18 | 19.894 | 11176 | 72.84 |
| 19 | 20.398 | 1633 | 8.57 |
| 20 | 20.715 | 6035 | 47.82 |
| 21 | 20.966 | 2572 | 11.76 |
| 22 | 22.252 | 4865 | 25.46 |
| 23 | 22.665 | 1416 | 4.66 |
| 24 | 22.902 | 2325 | 14.13 |
| 25 | 23.391 | 3295 | 13.08 |
| 26 | 23.813 | 6090 | 32.31 |
| 27 | 24.192 | 1248 | 4.1 |
| 28 | 24.705 | 4421 | 19.23 |
| 29 | 25.027 | 1602 | 10.13 |
| 30 | 25.323 | 18238 | 93.46 |
| 31 | 25.557 | 2961 | 21.14 |
| 32 | 25.897 | 1654 | 6.24 |
| 33 | 26.163 | 3158 | 26.16 |
| 34 | 26.527 | 4035 | 27.08 |
| 35 | 27.122 | 6309 | 30.05 |
| 36 | 27.903 | 1735 | 7.62 |
| 37 | 29.317 | 2979 | 24.86 |
| 38 | 29.556 | 1927 | 6.5 |
| 39 | 29.925 | 3141 | 25.09 |
| 40 | 30.706 | 2727 | 20.63 |
| 41 | 31.076 | 1826 | 10.78 |
| 42 | 32.862 | 1154 | 5.63 |
| 43 | 33.21 | 1419 | 13.83 |
| 44 | 34.19 | 1991 | 26.92 |
| 45 | 34.87 | 788 | 25.23 |
| 46 | 38.59 | 627 | 4.26 |
| 47 | 39.166 | 609 | 1.78 |
| 48 | 39.501 | 1366 | 9.92 |
| 49 | 39.674 | 620 | 2.45 |

### Example 6: Preparation of Form F of Compound 1

The preparation of Form F was carried out by contacting Form B of SKI-O-592 (100 g; Example 2) with water (300 mL) in a 3 L jacketed reactor at 20° C. to form a suspension. A solution of methane sulfonic acid (MSA, 2.5 eq.) in water (100 ml) was made in a separate round bottom flask and added to the jacketed reactor to give a clear solution. To the clear solution, at 20° C., was added acetone (400 ml), followed by seeding using Form F seed crystals (prepared previously by this procedure). The reaction mixture was stirred at 20° C. for 2 hours, following which additional acetone (1600 mL) was slowly added to the reactor over 10 h at 20° C. After complete addition of acetone, the reaction mixture was cooled to −10° C. at a cooling rate of 5° C./h and permitted to ripen at −10° C. for an additional 6 h. The suspension was filtered, and the filter cake dried under vacuum at 60° C. overnight (12 h).

XRPD analysis of the wet filter cake and solid obtained following vacuum drying gave an X-ray powder diffractogram pattern for Form F, as shown in FIG. 9. The observed 2θ±0.2 °2θ signals for Form F are summarized in Table 16. The purity of the dry solid was greater than 94.0% by HPLC analysis.

TABLE 16

| Signal | | Signal | Signal |
|---|---|---|---|
| No. | 2Theta(θ)° | Intensity | Intensity %. |
| 1 | 5.595 | 20080 | 43.88 |
| 2 | 8.880 | 2705 | 6.48 |
| 3 | 11.146 | 2158 | 5.05 |
| 4 | 12.38 | 1165 | 2.71 |
| 5 | 13.05 | 955 | 3.44 |
| 6 | 13.362 | 538 | 1.35 |
| 7 | 14.118 | 873 | 2.4 |
| 8 | 14.435 | 3482 | 7.29 |
| 9 | 15.987 | 2369 | 4.54 |
| 10 | 16.720 | 5044 | 15.22 |
| 11 | 17.597 | 1017 | 2 |
| 12 | 17.846 | 2615 | 4.86 |
| 13 | 18.057 | 1626 | 3.43 |
| 14 | 18.385 | 2103 | 6.9 |
| 15 | 18.886 | 40781 | 100 |
| 16 | 19.927 | 3358 | 7.47 |
| 17 | 20.285 | 4522 | 24.22 |
| 18 | 20.803 | 2592 | 4.68 |
| 19 | 21.010 | 1807 | 5.33 |
| 20 | 21.54 | 1081 | 2.93 |
| 21 | 22.100 | 1869 | 3.91 |
| 22 | 22.332 | 2575 | 5.76 |
| 23 | 22.709 | 3154 | 7.04 |
| 24 | 24.005 | 8615 | 19.4 |
| 25 | 24.49 | 863 | 1.35 |
| 26 | 25.455 | 2726 | 6.88 |
| 27 | 25.736 | 1632 | 6.88 |
| 28 | 26.042 | 1305 | 2.96 |
| 29 | 26.388 | 1972 | 4.38 |
| 30 | 26.832 | 1759 | 7.68 |
| 31 | 27.294 | 2559 | 5.46 |
| 32 | 27.84 | 468 | 0.9 |
| 33 | 28.125 | 2608 | 8.36 |
| 34 | 29.282 | 2309 | 13.88 |
| 35 | 30.81 | 1413 | 3.21 |
| 36 | 33.30 | 932 | 1.98 |
| 37 | 33.64 | 1032 | 3.19 |
| 38 | 34.323 | 1376 | 4.17 |
| 39 | 34.836 | 2406 | 7.74 |
| 40 | 35.634 | 1306 | 2.26 |
| 41 | 36.049 | 879 | 1.65 |
| 42 | 36.36 | 271 | 0.33 |
| 43 | 37.70 | 484 | 0.44 |

### Example 7: Synthesis of Form H of Compound 1

Early studies for synthesizing Form H explored different solvent systems and different crystallization methodologies. Table 17 summarizes the results of these experiments.

TABLE 17

| Target | Starting | | | |
|---|---|---|---|---|
| Form | Input | Solvents | Conditions | XRPD Results |
| Form H | Form F | MeOH/H₂O | Slurry | Form N + Form D |
| | | (95/5, v/v); 30 V | | |
| | | MeOH/H₂O | Ripening | Form F + Form A |
| | | (95/5, v/v); 30 V | | |
| | | EtOH/H₂O | | Form N + Form D |
| | | (95/5, v/v); 30 V | | |
| | | EtOH; 10 V | | Form J |
| | | MeOH; 10 V | | Form J |
| | | MeOH/H₂O | Evaporation | Form J + H |
| | | (9/1, v/v) | | |
| | | EtOH/H₂O | | Form H |
| | | (9/1, v/v) | | |
| | | THF/H₂O | | Form H |
| | | (9/1, v/v) | | |
| | | MeOH/H₂O | | Form H + |
| | | (95/5, v/v) | | monomesylate- |
| | | | | Form D |
| | | EtOH/H₂O | | Form J |
| | | (95/5, v/v) | | |

As indicated by the data in Table 17, Form H was obtained by slow evaporation at 25° C. Synthesis was carried out by contacting solid Form F of Compound 1 with each of the solvents or solvent mixtures shown in Table 18. The resultant suspension was stirred at room temperature for 3 days, following which the suspensions were filtered and the filtrate collected and subjected to slow evaporation at room temperature (~25° C.), by sealing the vessel comprising the filtrate with a sealing film and allowing the solvent to evaporate in a fume hood after puncturing the sealing film with a needle.

The solids obtained following slow evaporation of the filtrate were analyzed by XR-PD. Solvent mixtures comprising MeOH/H₂O=9/1 (v/v) or THF/H₂O=9/1 (v/v) gave Form H. XR-PD analysis of the solid obtained following slow evaporation and vacuum drying gave an X-ray powder diffractogram with signals for Form H, as shown in FIG. 11. The observed 2θ±0.2 °2θ signals for Form H are also shown in Table 18.

TABLE 18

| Signal | | Signal | Signal |
|---|---|---|---|
| No. | 2Theta(θ)° | Intensity | Intensity %. |
| 1 | 5.437 | 1895 | 23.93 |
| 2 | 8.722 | 2327 | 41.26 |
| 3 | 8.994 | 1006 | 11.66 |
| 4 | 9.588 | 610 | 9.16 |
| 5 | 11.077 | 545 | 10.62 |
| 6 | 12.75 | 1150 | 27.13 |
| 7 | 13.022 | 1425 | 7.93 |
| 8 | 13.432 | 1658 | 21.27 |
| 9 | 13.726 | 438 | 5.55 |
| 10 | 13.992 | 2416 | 28.65 |
| 11 | 14.302 | 896 | 13.29 |
| 12 | 14.511 | 1154 | 11.86 |
| 13 | 16.81 | 595 | 20.04 |
| 14 | 17.668 | 1706 | 11.23 |
| 15 | 18.249 | 1767 | 53.57 |
| 16 | 18.901 | 3780 | 100 |
| 17 | 19.234 | 997 | 8.34 |
| 18 | 19.882 | 3570 | 54.01 |
| 19 | 20.122 | 3145 | 49.57 |
| 20 | 20.61 | 857 | 26.87 |
| 21 | 22.05 | 1087 | 8.99 |
| 22 | 25.358 | 2992 | 84.6 |
| 23 | 25.808 | 2614 | 40.84 |
| 24 | 26.289 | 1340 | 26.68 |
| 25 | 26.671 | 751 | 8.52 |
| 26 | 27.130 | 647 | 6.16 |

### Example 8: Synthesis of Form J

Form J was synthesized by contacting SKI-O-592 (100 g, 1.0 eq.), with DMSO (500 ml) in a 3-liter jacketed reactor maintained at a temperature of 50° C. After stirring, a clear solution was obtained. A DMSO (300 ml) solution of methane sulfonic acid (MSA, 2.5 eq.) was added to the solution of SKI-O-592 in the reactor while maintaining the temperature of the reaction mixture at 50° C. The addition of MSA resulted in the formation of a clear yellow solution. To this solution was added water (120 ml) to produce a SKI-O-592/MSA solution followed by the addition of acetone (1600 ml), over a 10 h period. Once the addition of acetone was complete, the reaction mixture was cooled to 20° C., with stirring, at a rate of 5° C./hour, and the slurry allowed to ripen at 20° C. for an additional 3-6 hours. The cooled slurry was filtered to separate the solids from the filtrate and the wet solids were dried at 20° C. for 15 hours in an atmosphere with a relative humidity (RH) of 85%. The HPLC purity of the dried solid was 99.7%.

The procedure above produced crystals of Form J, and the procedure was subsequently repeated by adding seed crystals (2 wt %) of Form J, produced by the procedure, to the SKI-O-592/MSA solution, which was allowed to stir at 50° C. for an additional 2 hours (ripening). Remaining steps of the procedure were the same to produce further quantities of Form J.

The dried solid was analyzed by XRPD and gave a diffractogram with observed 2θ±0.2 °2θ signals summarized in Table 19 and shown in FIG. 13.

TABLE 19

| Signal | | Signal | Signal |
|---|---|---|---|
| No. | 2Theta(θ)° | Intensity | Intensity %. |
| 1 | 5.603 | 5203 | 26.77 |
| 2 | 8.562 | 5342 | 25.35 |
| 3 | 10.191 | 335 | 1.75 |
| 4 | 10.638 | 601 | 6.62 |
| 5 | 12.26 | 742 | 2.58 |
| 6 | 14.087 | 9097 | 51.77 |
| 7 | 14.604 | 1242 | 6.3 |
| 8 | 16.943 | 5439 | 36.6 |
| 9 | 17.188 | 2993 | 11.18 |
| 10 | 18.060 | 9951 | 51.68 |
| 11 | 18.346 | 4658 | 22.43 |
| 12 | 18.935 | 17331 | 100 |
| 13 | 19.306 | 3244 | 12.32 |
| 14 | 19.942 | 10569 | 54.95 |
| 15 | 20.571 | 4171 | 22.2 |
| 16 | 20.77 | 1063 | 7.15 |
| 17 | 21.024 | 3367 | 16.07 |
| 18 | 22.091 | 2655 | 10.75 |
| 19 | 22.526 | 1084 | 2.65 |
| 20 | 22.737 | 2461 | 10.74 |
| 21 | 22.998 | 818 | 3.6 |
| 22 | 23.360 | 2650 | 9.92 |
| 23 | 24.135 | 4408 | 19.03 |
| 24 | 24.585 | 3798 | 19.1 |
| 25 | 25.344 | 18341 | 98.13 |
| 26 | 25.901 | 2883 | 17.6 |
| 27 | 26.487 | 3691 | 29.07 |
| 28 | 27.04 | 830 | 3.44 |
| 29 | 27.257 | 4504 | 17.03 |
| 30 | 27.93 | 1274 | 4.97 |
| 31 | 29.452 | 2418 | 15.56 |
| 32 | 30.182 | 2151 | 14.82 |
| 33 | 31.036 | 2303 | 18.69 |
| 34 | 31.996 | 884 | 1.48 |
| 35 | 33.274 | 1376 | 15.7 |
| 36 | 34.218 | 1518 | 8.71 |
| 37 | 34.558 | 1097 | 10.06 |
| 38 | 35.58 | 721 | 9.24 |

### Example 9: Synthesis of Form L of Compound 1

Synthesis of Form L was carried out using a solvent/anti-solvent method. The starting material, Form F was dissolved, with stirring, at room temperature (25° C.) in DMSO (10 vol.). The resultant clear solution was contacted with an anti-solvent toluene (30 vol.). This resulted in the formation of a precipitate. The reaction mixture was stirred at room temperature, overnight (~15 hours), and then filtered. The precipitate was dried at 25° C. for ~10-15 hours and then analyzed by XRPD, HPLC, and ¹H-NMR.

XRPD analysis of the dry solid gave a diffractogram for Form L as shown in FIG. 15. The observed 2θ±0.2 °2θ signals shown for Form L are further summarized in Table 20.

TABLE 20

| Signal | | Signal | Signal |
|---|---|---|---|
| No. | 2Theta(θ)° | Intensity | Intensity %. |
| 1 | 5.532 | 27736 | 100 |
| 2 | 11.093 | 1723 | 11.42 |
| 3 | 14.03 | 685 | 4 |
| 4 | 16.680 | 4678 | 25.1 |
| 5 | 18.821 | 10050 | 62.65 |
| 6 | 20.744 | 707 | 13.8 |
| 7 | 22.270 | 2530 | 20.73 |
| 8 | 23.825 | 4036 | 35.62 |
| 9 | 25.00 | 290 | 3.12 |
| 10 | 25.38 | 718 | 5.23 |
| 11 | 29.54 | 505 | 4.33 |
| 12 | 30.681 | 825 | 4.48 |
| 13 | 35.00 | 981 | 23.26 |

### Example 10: Synthesis of Form M of Compound 1

The synthesis of Form M was carried out by vapor diffusion. A DMSO solution of Compound 1 at a concentration of 10-15 mg/ml, was charged into a glass tube and the tube comprising this solution was sealed using parafilm which was compromised by poking a few holes using a needle. This tube was then placed inside a second outer tube than contained an anti-solvent, such as ethyl acetate or toluene. The entire assembly was permitted to stand at room temperature for about 30 days, to permit the diffusion of the vapors of the anti-solvent into the DMSO solution of Compound 1.

The crystals thus formed were collected, washed with anti-solvent, and dried prior to analysis by single crystal X-ray diffraction (SCXRD). Table 21 lists the parameters for the unit cell of Form M based on SCXRD analysis.

TABLE 21

| Parameters | Results | |
|---|---|---|
| Crystal system | triclinic | |
| Space group | P-1 | |
| a, Å | 9.603 | (3) |
| b, Å | 9.959 | (5) |
| c, Å | 15.765 | (6) |
| α, deg | 95.50 | (2) |
| β, deg | 93.29 | (2) |
| γ, deg | 90.87 | (2) |
| V, Å3 | 1497.9 | (10) |

| | |
|---|---|
| Z | 42 |
| Dcalcd, g · cm-3 | 1.465 |
| T, K | 200 |
| Refl. | 3599/5400, |
| collected/unique | Rint = 0.089 |
| m, mm-1 | 2.181 |
| GOOF | 1.693 |

Alternatively, Form M was synthesized using Form J as starting material. Briefly, a vial containing solid Form J was sealed using parafilm. The sealing film was compromised using a needle to poke a few holes. This vial was then placed in a container where the relative humidity is maintained at 5% for a few hours. The solid material was then analyzed by XRPD. FIG. 18 shows the XRPD diffractogram and Table 22 summarizes the observed 2θ±0.2 °2θ signals for Form M.

TABLE 22

| Signal | | Signal |
|---|---|---|
| No. | 2Theta(θ)° | Intensity % |
| 1 | 5.62 | 14 |
| 2 | 8.77 | 7 |
| 3 | 12.98 | 8 |
| 4 | 13.77 | 9 |
| 5 | 14.50 | 8 |
| 6 | 14.73 | 30 |
| 7 | 15.01 | 17 |
| 8 | 16.26 | 52 |
| 9 | 16.91 | 13 |
| 10 | 17.82 | 26 |
| 11 | 18.16 | 52 |
| 12 | 18.39 | 37 |
| 13 | 18.81 | 21 |
| 14 | 19.02 | 20 |
| 15 | 19.71 | 28 |
| 16 | 19.87 | 97 |
| 17 | 20.38 | 62 |
| 18 | 20.77 | 8 |
| 19 | 21.01 | 27 |
| 20 | 21.68 | 7 |
| 21 | 22.09 | 18 |
| 22 | 22.32 | 30 |
| 23 | 22.63 | 45 |
| 24 | 23.19 | 8 |
| 25 | 23.50 | 81 |
| 26 | 24.62 | 8 |
| 27 | 25.01 | 15 |
| 28 | 25.11 | 17 |
| 29 | 25.49 | 43 |
| 30 | 25.76 | 17 |
| 31 | 26.00 | 100 |
| 32 | 26.38 | 10 |
| 33 | 26.97 | 15 |
| 34 | 27.30 | 7 |
| 35 | 27.69 | 25 |
| 36 | 29.31 | 7 |
| 37 | 29.68 | 16 |
| 38 | 29.87 | 14 |
| 39 | 30.43 | 13 |
| 40 | 30.88 | 3 |
| 41 | 32.37 | 7 |
| 42 | 33.28 | 9 |
| 43 | 33.91 | 6 |
| 44 | 34.18 | 6 |
| 45 | 34.51 | 6 |
| 46 | 34.88 | 6 |
| 47 | 35.14 | 5 |
| 48 | 35.85 | 11 |
| 49 | 36.06 | 10 |
| 50 | 36.64 | 6 |
| 51 | 37.58 | 3 |
| 52 | 38.06 | 8 |
| 53 | 39.48 | 3 |

### Example 11: Synthesis of Form N of Compound 1

Form N was obtained using Form J as starting material using a protocol like the one described above for Form M. Thus, Form N was obtained by placing Form J in a constant temperature and humidity chamber set at 25° C. and a relative humidity (RH) of 2000 for a period of 10 hours. The sample from the humidity chamber was analyzed by XRPD and gave a diffractogram with signals for Form N as shown in FIG. 19. Table 23 lists observed 2θ±0.2 °2θ signals characteristic for Form N. Form N was stored in a chamber with 10-20% RH.

TABLE 23

| Signal | | Signal | Signal |
|---|---|---|---|
| No. | 2Theta(θ)° | Intensity | Intensity %. |
| 1 | 5.712 | 67608 | 89.49 |
| 2 | 8.751 | 8511 | 14.1 |
| 3 | 10.523 | 1691 | 4.54 |
| 4 | 11.089 | 3179 | 5.8 |
| 5 | 11.363 | 2314 | 3.46 |
| 6 | 12.390 | 1378 | 1.72 |
| 7 | 12.979 | 585 | 0.82 |
| 8 | 13.759 | 3695 | 5.53 |
| 9 | 14.091 | 792 | 1.16 |
| 10 | 14.477 | 17633 | 28.06 |
| 11 | 15.033 | 6102 | 7.85 |
| 12 | 16.675 | 18859 | 25.17 |
| 13 | 17.058 | 16126 | 20.17 |
| 14 | 17.513 | 3956 | 5.14 |
| 15 | 17.803 | 2103 | 2.37 |
| 16 | 18.229 | 10441 | 17.14 |
| 17 | 18.389 | 4805 | 5.66 |
| 18 | 18.9011 | 34230 | 51.42 |
| 19 | 19.447 | 67929 | 100 |
| 20 | 20.249 | 10861 | 23.57 |
| 21 | 20.603 | 4178 | 6.54 |
| 22 | 20.768 | 7236 | 11.32 |
| 23 | 21.084 | 2118 | 3.39 |
| 24 | 21.324 | 4918 | 11.56 |
| 25 | 22.222 | 3939 | 6.6 |
| 26 | 22.590 | 5428 | 9.37 |
| 27 | 22.780 | 9244 | 13.75 |
| 28 | 22.978 | 6108 | 8.58 |
| 29 | 23.459 | 1833 | 1.95 |
| 30 | 24.147 | 24423 | 39.43 |
| 31 | 24.725 | 6011 | 6.86 |
| 32 | 24.910 | 5700 | 7.66 |
| 33 | 25.119 | 5478 | 7.79 |
| 34 | 25.676 | 20021 | 28.76 |
| 35 | 25.950 | 5578 | 15.23 |
| 36 | 26.243 | 4407 | 8.38 |
| 37 | 26.965 | 2722 | 5.66 |
| 38 | 27.216 | 1958 | 3.21 |
| 39 | 27.641 | 5535 | 8.01 |
| 40 | 28.387 | 1158 | 3.85 |
| 41 | 29.757 | 1881 | 8.82 |
| 42 | 30.303 | 1187 | 2.38 |
| 43 | 30.639 | 7166 | 12.46 |
| 44 | 31.095 | 1150 | 1.25 |
| 45 | 31.47 | 847 | 1.11 |
| 46 | 32.038 | 3894 | 6.3 |
| 47 | 32.251 | 665 | 0.79 |
| 48 | 33.143 | 1107 | 1.45 |
| 49 | 33.712 | 999 | 2.9 |
| 50 | 34.01 | 376 | 0.47 |
| 51 | 34.350 | 2411 | 8.75 |
| 52 | 34.793 | 2492 | 5.44 |
| 53 | 35.214 | 1193 | 3.33 |
| 54 | 35.889 | 2115 | 3.21 |
| 55 | 36.235 | 3635 | 10.07 |
| 56 | 36.463 | 1108 | 1.16 |
| 57 | 38.253 | 919 | 1.16 |
| 58 | 38.607 | 538 | 0.64 |
| 59 | 39.105 | 387 | 0.99 |
| 60 | 39.414 | 470 | 0.68 |

Form N was also obtained by contacting the Form B of SKI-O-592 with acetone (solution “A”, 12.5 vol.). A solution of methane sulfonic acid (MSA, 2.5 eq.) in acetone (12.5 vol.) was added at room temperature with stirring to solution “A”. The resultant reaction mixture was stirred at room temperature (20-25° C.) for a few minutes and then heated to 50° C. The reaction mixture is maintained at 50° C. for 2 hours and then cooled gradually to room temperature at a rate of 5° C./hour. The cooled reaction is stirred overnight and then centrifuged to separate the solids from the filtrate. The wet solids were analyzed by XRPD (30-40% RH) and showed a diffractogram that conforms to Form N.

Alternatively, Form N was synthesized by dissolving Compound 1 in methanol/water (95/5 v/v, 100 vol.) solvent. The resultant solution was filtered, and the solvent evaporated at 50° C. XRPD analysis of the resultant solid showed a diffractogram that conforms to Form N.

### Example 12: Impurity Spiking Studies

The synthesis of SKI-O-592 and SKI-O-703 produces two impurities, namely Compound 4 (703-C09) and Compound 3 (703-C11), structurally shown below.

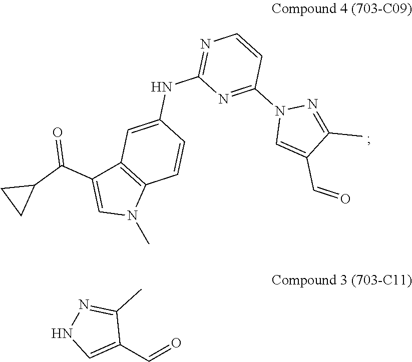

Thus, it was necessary to investigate and develop experimental conditions for removing these impurities. To accomplish this, the present inventors performed impurity spiking studies to evaluate and optimize the solvent and/or solvent mixtures, as well as a process for removing these impurities to produce pure, pharmaceutical grade crystalline forms of SKI-O-592 and Compound 1. Table 24 summarizes the data from early studies using two solvent systems—acetone/water and DMSO/acetone/water.

TABLE 24

| | | 703-C06 | 703-C11 |
|---|---|---|---|
| Process Condition | | (Compound 2) | (Compound 3) |
| Acetone/H2O | SKI-O-592 | <7.5 ppm | ND |
| | | (LOQ = 7.5 ppm) | |
| | SKI-O-703 | 0.33 ppm | 2.5 ppm |
| | | (LOD = 0.9 ppm) | (LOQ = 2.25 ppm) |
| DMSO/ | SKI-O-703 | <0.9 ppm | <0.18 ppm |
| Acetone/H2O | | (LLOQ = 0.9 ppm) | (LLOQ = 0.18 ppm) |

ND: Not Detected

Table 25 shows XRPD and purity data from studies at the 10 g scale. HPLC was used to analyze purity of the test samples, and the method used had a lower limit of detection of 0.02%, for both impurities.

TABLE 25

| | | Seed | | | | | | |
|---|---|---|---|---|---|---|---|---|
| Crystallization | MSA | Crystal | | Form by | HPLC Results | | | |
| Solvent | Used | Used | | XRPD | SKI-O-703 | C09 | C11 | Yield |
| DMSO/Acetone/H₂O | 2.5 eq | Form J | Input | SKI-O- | 93.5% | 5.6% | 0.6% | N/A |
| (8 V/16 V/1.2 V) | | | | 592 | | | | |
| | | | Product | Form J | 99.1% | 0.9% | 0.0% | 90% |
| | | | Purgeability | N/A | N/A | 83.7% | 100.0% | N/A |
| | | | Input | SKI-O- | 98.0% | 1.3% | 0.6% | N/A |
| | | | | 592 | | | | |
| | | | Product | Form J | 99.7% | 0.2% | 0.0% | 92% |
| | | | Purgeability | N/A | N/A | 80.7% | 100.0% | N/A |
| Acetone/H₂O | 2.5 eq | Form F | Input | SKI-O- | 93.1% | 5.8% | 0.6% | N/A |
| (20 V/4 V) | | | | 592 | | | | |
| | | | Product | Form F | 94.5% | 5.4% | 0.0% | 92% |
| | | | Purgeability | N/A | N/A | 8.0% | 100.0% | N/A |
| | | | Input | SKI-O- | 98.0% | 1.3% | 0.6% | N/A |
| | | | | 592 | | | | |
| | | | Product | Form F | 99.0% | 1.0% | 0.0% | 93% |
| | | | Purgeability | N/A | N/A | 24.5% | 100.0% | N/A |

N/A: Not Available

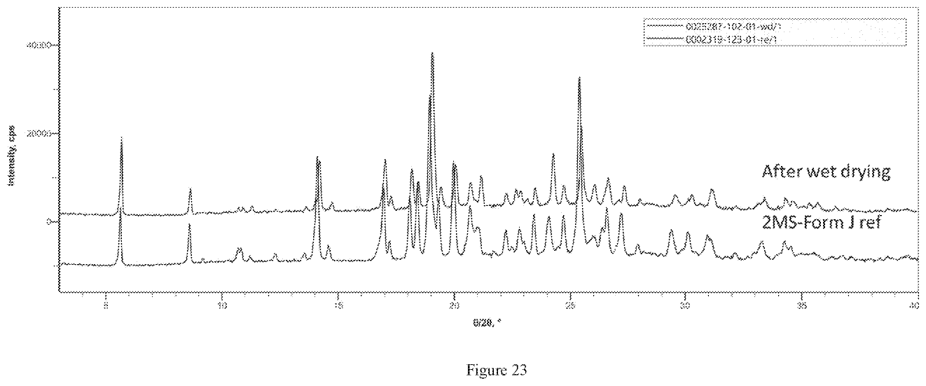

Table 25 illustrates that a mixed solvent system of DMSO/Acetone/Water (8V/16V/1.2V) can be used to obtain Form J in high purity, greater than 99%, using Form J as seed crystals. XRPD diffractogram signals of the solid product are shown in FIG. 23 and confirm that the product obtained is Form J.

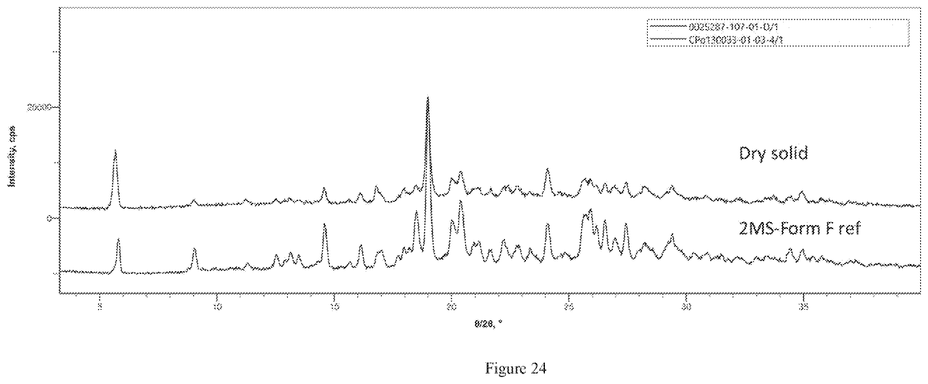

High purity Form F, however, was obtained using an acetone/water (20V/4V) solvent system. First, the starting material freebase Form B of Compound 1, was dissolved in water (3.0 vol.) to obtain a slurry which was spiked with C09 (5%) and C11 (0.5%) impurities prior to contacting the mixture with an aqueous solution of methane sulfonic acid (2.5 eq., 4 vol. water). This solution was heated to 30° C., cooled and then seeded by crystalline Form F. Acetone (20 vol.) was then added to the reaction mixture which was stirred at room temperature (20° C.), to initiate precipitation. The solid precipitate was then filtered and dried. XRPD analysis was carried out for both the wet solid precipitate as well as the dry solid and gave a diffractogram with signals shown in FIG. 24. HPLC analysis was used to quantify the purity of the solid and the results are shown in Table 25.

### Example 13: Intrinsic Dissolution Studies

Intrinsic dissolution rate (IDR) refers to the rate at which a pure drug or crystal form dissolves in a specific medium (e.g., solvent) under controlled conditions. IDR specifically measures the speed of the drug releasing from its solid form into a solution. The present inventors measured the intrinsic dissolution rates of Forms B, F, J, and N, of Compound 1 over a 10-minute time interval. The IDR's of these crystalline forms were compared to amorphous Compound 1.

IDR studies were carried out as follows: 80 mg of the test form was subjected to a pressure of 10 MPa for 1 minute to prepare a disc. The disc preparation was carried out using relative humidity conditions at which each test form is known to be stable.

Table 26 summarizes the data from a study that measured the IDR using discs of each of these test samples. These data show that the intrinsic dissolution rate of crystal Forms J and N are greater than the IDR for Forms B and F as well as amorphous form used here for comparative purposes.

TABLE 26

| Time | | | | | Amor- |
|---|---|---|---|---|---|
| point | Form N | Form J | Form F | Form B | phous |
| (min) | (mg/cm²) | (mg/cm²) | (mg/cm²) | (mg/cm²) | (mg/cm²) |
| 0 | 0.0 | 0.0 | 0.0 | 0.0 | 0.0 |
| 2 | 74.2 | 90.6 | 43.6 | 57.9 | 65 |
| 5 | 177.3 | 164.6 | 127 | 126.8 | 155.1 |
| 7 | 228.6 | 205.1 | 167.9 | 183.9 | 206.7 |
| 10 | 292.2 | 274.6 | 239.9 | 230.5 | 280.5 |
| IDR (mg/ | 29.5 | 26.4 | 24.2 | 23.4 | 28.1 |
| min/cm²) | | | | | |
| % vs | 105% | 94% | 86% | 83% | 100% |
| Amorphous | | | | | |

### Example 14: Pharmacokinetic Study

Plasma pharmacokinetics (PK) was investigated using male SD rats (N=3/group) after orally administering capsules comprising Form B, Form F, Form J, Form N, or Amorphous form to the test animals.

### Animals

Male SD Rats were purchased from Zhejiang Vital River Laboratory Animal Technology Co., Ltd. The animals were 6-8 weeks old, with body weights of 228-251 grams on the day of dosing. All animals for PO were fasted overnight prior to dosing and were fed approximately 4 hours after dosing. The PK study was approved by the Pharmaron Institutional Animal Care and Use Committee (IACUC).

### Dosing Procedure

Rats were dosed using oral gavage. One gastric capsule (Size 9) each containing 8.75 mg of Form B, Form F, Form J, Form N, or amorphous form based on animal weight of 250 g were administered by oral gavage syringe.

### Sample Collection

Blood samples (0.2 mL) were collected from each animal via the jugular vein at the following time points: 0.5 h, 1 h, 2 h, 4 h, 8 h, 12 h and 24 h post-dose. Blood samples were placed into tubes containing Heparin sodium, and then centrifuged at 4000 g for 5 minutes at 4° C. to obtain plasma. All samples were stored at −75±15° C. until LC-MS/MS analysis.

### Preparation of Standard Solutions Solution for LC-MS/MS Analysis

A solution of SKI-O-592 (free form) was prepared in DMSO by vortexing a 1 mg/mL solution to make the standard stock. Calibration standard working solutions were prepared at concentrations of 5, 10, 20, 50, 100, 500, 1000, 5000, and 10000 ng/mL by serial dilution of the standard stock solution using 50% acetonitrile in water.

Quality control working solutions at concentrations of 10, 20, 50, 500, 4000 and 8000 ng/mL were prepared by serial dilution of the standard stock solution using 50% acetonitrile in water. These QC samples were prepared on the day of analysis in the same way as calibration standards. SKI-O-592-D3 (free form) was prepared in DMSO by vortexing a 1 mg/mL solution to make an internal standard stock solution.

### Sample Treatment

5 μL of each calibration standard working solution (5, 10, 20, 50, 100, 500, 1000, 5000, and 10000 ng/mL) was added to 50 μL of blank SD Rats plasma to obtain calibration standards of 0.5-1000 ng/mL (0.5, 1, 2, 5, 10, 50, 100, 500, and 1000 ng/mL) in a total volume of 55 μL. Quality Control (QC) samples were prepared independently from solutions used for the calibration curves, at 1 ng/mL (low-1), 2 ng/mL (low-2), 5 ng/mL (low-3), 50 ng/mL (mid), 400 ng/mL (high-1) and 800 ng/mL (high-2) using blank plasma were prepared.

55 μL of standards, 55 μL of QC samples or 55 μL of unknown samples (50 μL of plasma sample with 5 μL 50% acetonitrile in water) were mixed with 200 μL of acetonitrile containing IS (SKI-O-592-D3 with concentration at 20 ng/mL) to precipitate proteins. The resultant mixtures were then vortexed for 30 sec. After centrifugation at 4° C., 3900 rpm for 15 min, the supernatant was separated and diluted at a ratio of 1:2 (v/v) with water. 4 μL of diluted supernatant was injected into the LC-MS/MS for quantitative analysis.

### LC-MS/MS Conditions

The LC-MS/MS system consisted of Degasser DGU-20A5R, S, Liquid Chromatograph LC-30AD, Communications Bus Module CBM-20A, Auto Sampler SIL-30AC, Rack changer II and an AB API 5500 LC/MS/MS instrument (Serial No. EF20351803).

Chromatographic separation was performed on a HALO C18 90A 5 μm (50*2.1 mm) at room temperature. The mobile phase was composed of: Solvent A—5% acetonitrile (0.1% formic acid) in water; Solvent B—95% acetonitrile (0.1% formic acid) in water. The flow rate was 0.6 mL/min., and the injection volume was 4 μL.

Positive mode electrospray ionization (ESI) was performed on a Turbo V® ion source to obtain a protonated ion of SKI-O-592 (RS) and SKI-O-592-D3 (IS). A multiple reaction monitoring (MRM) method was selected for quantitative analysis. The optimized transitions were 473.77/385.10 and 476.97/388.00 for SKI-O-592 and SKI-O-592-D3, respectively. The instrument parameters were set as follows: ion spray voltage: 5500 V; curtain gas: 35 psi; nebulizer gas: 50 psi; turbo gas: 50 psi; collision gas: 9 psi; temperature: 500° C. Compound dependent parameters, optimized for the MRM method, are shown in Table 27 below.

TABLE 27

| | SKI-O-592 | SKI-O-592-D3 |
|---|---|---|
| Compound ID | (RS) | (IS) |
| Transition | 473.77/385.10 | 476.97/388.00 |
| DP (de-clustering | 71 | 71 |
| potential) | | |
| CE (collision energy) | 25 | 23 |
| CXP (collision cell | 10 | 10 |
| exit potential) | | |

### Data Acceptance Criteria

### Acceptance Criteria of Standard Calibration Samples:

At least 6 samples were analyzed to obtain a calibration curve. Acceptance of calibration standards requires calculated concentration to be within 80%-120% of the nominal concentration. Additionally, 75% of the calibration standards should be within the acceptable range.

### Acceptance Criteria of Quality Control Samples:

At least 3-independent concentrations of the quality control sample (QCs) were analyzed in each run. Two individual samples were analyzed for each concentration. Acceptance criteria for QC samples requires the calculated concentration to be within 80%-120% of the nominal concentration. QCs should be analyzed amongst all unknown samples and ⅔ of the QCs should be within the acceptable range, and at least 1 sample at each concentration should be within the acceptable range.

### Statistical Analysis

Data acquisition was performed by Sciex Analyst 1.7.2 software (AB Sciex, Forster City, CA). All concentration data and Pharmacokinetic parameters were reported to 3 significant figures. BLOQ was set to zero in calculation. Data statistics were performed using Excel 2010 software.

### Pharmacokinetic Analysis

SKI-O-592 plasma concentrations for each animal following oral administration were used to calculate pharmacokinetic parameters by employing a non-compartmental analysis (Phoenix TM WinNonlin® 8.3). The linear trapezoidal algorithm was used for AUC calculation. Plasma pharmacokinetic parameters.

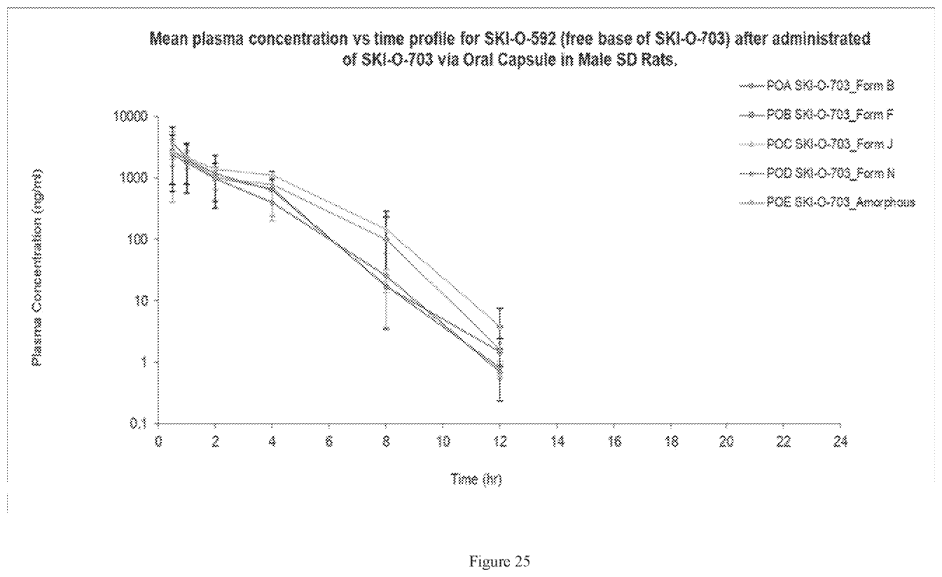

The pharmacokinetics of different crystalline forms of Compound 1, namely, Forms B, F, J, and N as well as amorphous form, were evaluated for each dosing group of male SD Rats (N=3/Group), and the data relating plasma concentrations versus time after oral administration of the different crystalline forms of Compound 1 are graphically shown in FIG. 25 and represented in Table 2.

We claim:

1. A monohydrate crystalline Form J of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), characterized by an X-ray powder diffraction (XRPD) pattern, comprising signals at 14.1, 16.9, 18.1, 19.9, 15.3, and 26.5 2θ±0.2 °2θ as determined on a diffractometer using Cu-Kα radiation at a wavelength of 1.54 Å.

2. The monohydrate crystalline Form J of Compound 1 according to claim 1, wherein the XRPD pattern is substantially as shown in FIG. 13.

3. The monohydrate crystalline Form J of Compound 1 according to claim 1, characterized by a thermogravimetric analysis (TGA) thermogram comprising a weight loss of about 2.5% between 3° and 100° C.

4. The monohydrate crystalline Form J of Compound 1 according to claim 3, wherein the TGA thermogram is substantially as shown in FIG. 14.

5. The monohydrate crystalline Form J of Compound 1 according to claim 1, comprising between 0.01 ppm to 0.9 ppm of 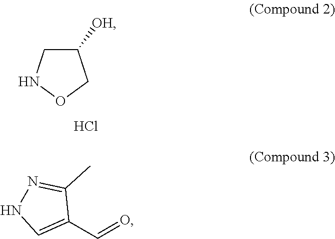
and combinations thereof.

6. The monohydrate crystalline Form J of Compound 1 according to claim 1, comprising 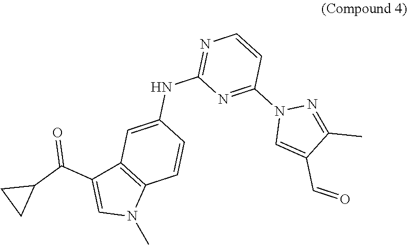 [SMILES: Cc1nn(-c2ccnc(Nc3ccc4c(c3)c(C(=O)C3CC3)cn4C)n2)cc1C=O]
in a weight percentage amount of about 0.05% to about 0.25%.

7. A monohydrate crystalline Form F of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), characterized by an X-ray powder diffraction (XRPD) pattern comprising signals at 5.6, 16.7, 18.9, 20.3, 24.0, and 29.3 °2θ±0.2 θ2θ as determined using a diffractometer using Cu-Kα radiation at a wavelength of 1.54 Å.

8. The monohydrate crystalline Form F of Compound 1 according to claim 7, wherein the XRPD pattern is substantially as shown in FIG. 9.

9. The monohydrate crystalline Form F of Compound 1 according to claim 7, characterized by a thermogravimetric analysis (TGA) thermogram comprising a weight loss of about 2.6% between 3° and 100° C.

10. The monohydrate crystalline Form F of Compound 1 according to claim 9, wherein the TGA thermogram is substantially as shown in FIG. 10.

11. A partial hydrate crystalline Form N of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), characterized by an X-ray powder diffraction (XRPD) pattern comprising signals at 14.5, 16.7, 17.1, 19.4, 20.2, 24.1, and 25.7 °2θ±0.2 °2θ as determined using a diffractometer using Cu-Kα, radiation at a wavelength of 1.54 Å.

12. The partial hydrate crystalline Form N of claim 11, wherein the XRPD pattern is substantially as shown in FIG. 19.

13. The partial hydrate crystalline Form N of Compound 1 according to claim 11, characterized by a thermogravimetric analysis (TGA) thermogram comprising a weight loss of about 1.0% between 3° and 100° C.

14. The partial hydrate crystalline Form N of Compound 1 according to claim 13, wherein the TGA thermogram is substantially as shown in FIG. 20.

15. An anhydrate crystalline Form M of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), characterized by an X-ray powder diffraction (XRPD) pattern comprising signals at 16.3, 18.2, 19.9, 20.4, 23.5, and 26.0 °2θ±0.2 °2θ as determined using a diffractometer using Cu-Kα, radiation at a wavelength of 1.54 Å.

16. The anhydrate crystalline Form M of Compound 1 according to claim 15, wherein the XRPD pattern is substantially as shown in FIG. 18.

17. Crystalline Form E of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), characterized by an X-ray powder diffraction (XRPD) pattern substantially as shown in FIG. 7.

18. Crystalline Form E of Compound 1 according to claim 17, characterized by a TGA thermogram comprising a weight loss of about 1.8% that between 3° and 100° C.

19. Crystalline Form E of Compound 1 according to claim 18, wherein the TGA thermogram is substantially as shown in FIG. 8.

20. A pharmaceutical composition comprising (i) a therapeutically effective amount of monohydrate crystalline Form J according to claim 1, monohydrate crystalline Form F according to claim 7, partial hydrate crystalline Form N according to claim 11, anhydrate crystalline Form M according to claim 15, or crystalline Form E according to claim 17, and (ii) a solid pharmaceutically acceptable excipient.

21. A method for treating a subject suffering from idiopathic thrombocytopenic purpura (ITP), rheumatoid arthritis (RA), warm autoimmune hemolytic anemia (wAIHA), systemic lupus erythematosus, Psoriasis, or antiphospholipid syndrome (APS), comprising administering to the subject a therapeutically effective amount of monohydrate crystalline Form J according to claim 1, monohydrate crystalline Form F according to claim 7, partial hydrate crystalline Form N according to claim 11, anhydrate crystalline Form M according to claim 15, or crystalline Form E according to claim 17.

22. A method for synthesizing crystalline Form J of cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone bis-mesylate (“Compound 1”), comprising:
(a) dissolving cyclopropyl-[5-[[4-[4-[[(4S)-4-hydroxy-1,2-oxazolidin-2-yl]methyl]-3-methylpyrazol-1-yl]pyrimidin-2-yl]amino]-1-methylindol-3-yl]methanone in warm DMSO whereby a solution is formed;
(b) contacting the solution from (a) with a DMSO solution of methanesulfonic acid whereby a second solution is formed;
(c) adding water and then acetone to the second solution from (b) whereby a third solution is formed; and
(d) cooling the third solution from (c) whereby Form J is formed.

23. The method according to claim 22, wherein the solution in (a) is at about 50° C.

24. The method according to claim 22, wherein the second solution in (b) is at about 50° C.

25. The method according to claim 22, wherein the cooling in (d) occurs at a rate of about 5° C./h.
